SEQUENCE LISTING (10 sequences)

SEQ ID NO: 1 - DNA, 20 bp, Artificial
  synthetic primer
ctagcctgggcaacaaacga

SEQ ID NO: 2 - DNA, 20 bp, Artificial
  synthetic primers
gcaggaagcaggaatggaac

SEQ ID NO: 3 - DNA, 20 bp, Artificial
  synthetic primer
taggagatggattattggtg

SEQ ID NO: 4 - DNA, 20 bp, Artificial
  synthetic primer
aagcaactttgcaatgagtg

SEQ ID NO: 5 - DNA, 21 bp, Artificial
  synthetic primers
cctggtctaggaagagtgtca

SEQ ID NO: 6 - DNA, 23 bp, Artificial
  synthetic primer
gtgtaagcatctgtgtatactac

SEQ ID NO: 7 - DNA, 20 bp, Artificial
  synthetic primer
ctatcaataagcattggcct

SEQ ID NO: 8 - DNA, 19 bp, Artificial
  synthetic primer
ctggagttgagactagcct

SEQ ID NO: 9 - DNA, 20 bp, Artificial
  synthetic primer
ctgcgctttgcactattggg

SEQ ID NO: 10 - DNA, 27 bp, Artificial
  synthetic primer
gtgtcttcgggcagcatagcattgcct

(19) United States
(12) Patent Application Publication
(10) Pub. No.: US 2010/0285977 A1
(43) Pub. Date: Nov. 11, 2010

(54) DIFFERENTIATION BETWEEN BRCA1-ASSOCIATED AND SPORADIC TUMOURS

(75) Inventors: Petra Marleen Nederlof, Amsterdam (NL); Franciscus Bartholomeus Laurentius Hogervorst, Amsterdam (NL); Senno Verhoef, Heiloo (NL)

Correspondence Address: BROWDY AND NEIMARK, P.L.L.C.;624 NINTH STREET, NW, SUITE 300, WASHINGTON, DC, 20001-5303, US

(73) Assignee: Het Nederlands Kanker Instituut, Amsterdam (NL)

(21) Appl. No.: 12/682,704

(22) PCT Filed: Oct. 9, 2008
(86) PCT No.: PCT/NL08/50641

§ 371 Date: Jul. 19, 2010

(30) Foreign Application Priority Data
Oct. 11, 2007 (EP) 07118284.4

(51) Int. Cl. C40B 30/00; C12Q 1/68; C40B 40/06

(52) U.S. Cl. 506/7; 435/6; 506/16

(57) ABSTRACT

The present invention relates to methods for classifying a sample as from a BRCA1-related tumour or from a sporadic tumour based on copy number analysis of a numbers of genomic locations. The invention further provides sets of nucleic acid probes, arrays and kits comprising these sets and arrays, for use in the diagnostic and/or prognostic methods of the invention for tumour classification.

### FIELD OF THE INVENTION

The present invention concerns the fields of medicin and tumour molecular biology. In particular the invention relates to means and methods for diagnosis and prognosis of BRCA1-like tumours using tumour classification based on specific genomic copy number alterations (CNA) by techniques such as array CGH.

### BACKGROUND OF THE INVENTION

Breast cancer is the most common cancer in the developed countries and one of the leading causes of death in women, one out of every nine women will be affected by breast cancer (Weir et al., 2003, J Natl Cancer Inst. 95(17):1276-99; ACS 2003-2004). Approximately 10-15% of the breast cancer cases show a positive family history for breast cancer (Anton-Culver et al., 1996, Genet Epidemiol. 13(2):193-205), and of those approximately 25-50% is due to a mutation in the breast cancer predisposition genes BRCA1 and BRCA2 (Narod and Foulkes, 2004, Nat Rev Cancer. 4(9):665-76). Women carrying a mutation in BRCA1/2 have a lifetime risk of breast cancer up to 80% (Ford 1998, Easton 1995, Antoniou 2003, King 2003). Identification of a BRCA1/2 mutation in a patient may not only influence the treatment of a patient (e.g. radiation, bilateral prophylactic mastectomy (Tercyak 2006), or oophorectomy) and surveillance (tumour prevention), but also allows pre-symptomatic mutation screening of family members.

Based on family history and age of onset, breast cancer patients are eligible for DNA screening for pathogenic mutations in BRCA1/2. Diagnostics currently includes mutation scanning and sequencing of gene fragments in germ line DNA. All these techniques do have their disadvantages and a part of the mutations remain undetected (Van der Hout 2006). It is estimated that in 20-30% of the BRCA1-linked families no mutation is found (Narod 1995, Ford 1998). Additionally, the detection of variants of unknown clinical significance complicates counselling and clinical management. Therefore, an additional tool that would indicate BRCA1 and BRCA2 involvement in breast cancer would be an asset to the current clinical diagnostics.

Numerous studies show specific genetic characteristics on which tumours can be categorised in subclasses. Due to the diversity of tumours, in many cases multiple features, i.e. characteristics, are needed to be able to distinguish between these subclasses. An objective method that would be able to discriminate between tumour types could help counselees and clinicians in their decision of treatment (van 't Veer 2002 Nature. 415(6871):530-6; Hannemann 2006, Breast Cancer Res. 8(5):R61). For hereditary BRCA1 cancer, previous publications from us and others show that these tumours develop distinct genetic alterations on which they can be recognised and be distinguished from non-hereditary, i.e. sporadic, tumours. Various methods using expression profiling (Hedenfalk 2001) or comparative genomic hybridisation (CGH) (Wessels 2002, Van Beers 2005, Jonsson 2005) show specific genetic alterations for these tumour groups. Although tumour mRNA has led to many molecular portraits, fresh frozen tissue is not often available especially when family screening includes diseased relatives. Formalin fixation and embedding in paraffin (FFPE) on the other hand is the common procedure for all hospitals to store tumour tissue. To perform CGH studies, we have previously shown that paraffin embedded tumours are of adequate quality (Van Beers 2006). The enhanced resolution of a micro array, compared with metaphase CGH (Wessels 2002), can improve the sensitivity and specificity of the detection of BRCA1 or BRCA2 tumours using CGH technology. Additionally, it will also provide a better estimate of the location of the chromosomal breakpoints of the genetic aberrations.

Due to the large numbers of families and individuals that are eligible for DNA-screening, it is not feasible to extend the current diagnostic assays beyond the current offered tests and screen all family members. Also, the risk for carrying a mutation calculated using prediction models that are based on family history are often worse predictors, especially when it comes to small families. An objective pre-screening test, based on the tumour only, that would indicate the involvement of BRCA1 in a family could help to select only those patients who need more extensive analysis. Genomic profiling of tumours, using comparative genomic hybridisation, could be such a strategy, however, this approach has not been validated earlier in a diagnostic setting.

It is an object of the present invention to provide for a method and means for prognostic and/or diagnostic genomic profiling of tumours for BRCA1 involvement.

### DESCRIPTION OF THE INVENTION

### Definitions

The term “hybridisation” refers to the binding of two single stranded nucleic acids via complementary base pairing. The terms “hybridizing specifically to”, “specific hybridization”, and “selectively hybridize to,” as used herein refer to the binding, duplexing, or hybridizing of a nucleic acid molecule preferentially to a particular nucleotide sequence under stringent conditions. The term “stringent conditions” refers to conditions under which a probe will hybridize preferentially to its target subsequence, and to a lesser extent to, or not at all to, other sequences in a mixed population (e.g., a cell lysate or DNA preparation from a tissue biopy) A “stringent hybridization” and “stringent hybridization wash conditions” in the context of nucleic acid hybridization (e.g., as in array, Southern or northern hybridizations) are sequence dependent, and are different under different environmental parameters. An extensive guide to the hybridization of nucleic acids is found in, e.g., Tijssen (1993) Laboratory Techniques in Biochemistry and Molecular Biology—Hybridization with Nucleic Acid Probes part I, Ch. 2, “Overview of principles of hybridization and the strategy of nucleic acid probe assays,” Elsevier, N.Y. (“Tijssen”). Generally, highly stringent hybridization and wash conditions are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleic acids which have more than 100 complementary residues on an array or on a filter in a Southern or northern blot is 42° C. using standard hybridization solutions (see, e.g., Sambrook and Russell (2001) Molecular Cloning: A Laboratory Manual (3rd ed.) Vol. 1-3, Cold Spring Harbor Laboratory, Cold Spring Harbor Press, NY, and detailed discussion, below), with the hybridization being carried out overnight. An example of highly stringent wash conditions is 0.15 M NaCl at 72° C. for about 15 minutes. An example of stringent wash conditions is a 0.2×SSC wash at 65° C. for 15 minutes (see, e.g., Sambrook supra. for a description of SSC buffer). Often, a high stringency wash is preceded by a low stringency wash to remove background probe signal. An example of a medium stringency wash for a duplex of, e.g., more than 100 nucleotides, is 1×SSC at 45° C. for 15 minutes. An example of a low stringency wash for a duplex of, e.g., more than 100 nucleotides, is 4× to 6×SSC at 40° C. for 15 minutes.

The term “nucleic acid” or “polynucleotide” as used herein refers to a deoxyribonucleotide or ribonucleotide in either single- or double-stranded form. The term encompasses nucleic acids containing known analogues of natural nucleotides which have similar or improved binding properties, for the purposes desired, as the reference nucleic acid. The term also includes nucleic acids which are metabolized in a manner similar to naturally occurring nucleotides or at rates that are improved for the purposes desired. The term also encompasses nucleic-acid-like structures with synthetic backbones. DNA backbone analogues provided by the invention include phosphodiester, phosphorothioate, phosphorodithioate, methylphosphonate, phosphoramidate, alkyl phosphotriester, sulfamate, 3′-thioacetal, methylene(methylimino), 3′-N-carbamate, morpholino carbamate, and peptide nucleic acids (PNAs); see Oligonucleotides and Analogues, a Practical Approach, edited by F. Eckstein, IRL Press at Oxford University Press (1991); Antisense Strategies, Annals of the New York Academy of Sciences, Volume 600, Eds. Baserga and Denhardt (NYAS 1992); Milligan (1993) J. Med. Chem. 36:1923-1937; Antisense Research and Applications (1993, CRC Press). PNAs contain non-ionic backbones, such as N-(2-aminoethyl)glycine units. Phosphorothioate linkages are described in WO 97/03211; WO 96/39154; Mata (1997) Toxicol. Appl. Pharmacol. 144:189-197. Other synthetic backbones encompassed by the term include methyl-phosphonate linkages or alternating methylphosphonate and phosphodiester linkages (Strauss-Soukup (1997) Biochemistry 36: 8692-8698), and benzylphosphonate linkages (Samstag (1996) Antisense Nucleic Acid Drug Dev 6: 153-156).

The term “array”, “micro-array”, “nucleic acid array” and “biochip” are used herein interchangeably. They refer to an arrangement, on a substrate surface, of multiple nucleic acid molecules of predetermined identity, of which preferably the sequences are known. Each nucleic acid molecule is immobilized to a “discrete spot” (i.e., a defined location or assigned position) on the substrate surface. The term “micro-array” more specifically refers to an array that is miniaturized so as to require microscopic examination for visual evaluation. The arrays used in the methods of the invention are preferably microarrays. The nucleic acid array as used herein is a plurality of target elements, each target element comprising one or more nucleic acid molecules (probes) immobilized on one or more solid surfaces to which sample nucleic acids can be hybridized. The nucleic acids of a probe can contain sequence(s) from specific genes or clones, e.g. from specific genomic regions described in Table 1. Other probes may contain, for instance, reference sequences. The probes of the arrays may be arranged on the solid surface at different densities. The probe densities will depend upon a number of factors, such as the nature of the label, the solid support, and the like. One of skill will recognize that each probe may comprise a mixture of nucleic acids of different lengths and sequences. Thus, for example, a probe may contain more than one copy of a cloned piece of DNA or RNA, and each copy may be broken into fragments of different lengths. The length and complexity of the nucleic acid fixed onto the target element is not critical to the invention. One of skill can adjust these factors to provide optimum hybridization and signal production for a given hybridization procedure, and to provide the required resolution among different genes or genomic locations.

The term “probe” or “nucleic acid probe”, as used herein, is defined to be one or more nucleic acid fragments whose specific hybridization to a sample can be detected. The probe may be unlabelled or labelled as described below so that its binding to the target or sample can be detected. The probe is produced from a source of nucleic acids from one or more particular (preselected) portions of a chromosome, e.g., one or more clones, an isolated whole chromosome or chromosome fragment, or a collection of polymerase chain reaction (PCR) amplification products. The probes of the present invention are produced from nucleic acids found in the regions described herein.

The probe may also be isolated nucleic acids immobilized on a solid surface (e.g., nitrocellulose, glass, quartz, fused silica slides), as in an array. In some embodiments, the probe may be a member of an array of nucleic acids as described, for instance, in WO 96/17958. Techniques capable of producing high density arrays can also be used for this purpose (see, e.g., Fodor (1991) Science 767-773; Johnston (1998) Curr. Biol. 8: R171-R174; Schummer (1997) Biotechniques 23: 1087-1092; Kern (1997) Biotechniques 23: 120-124; U.S. Pat. No. 5,143,854). One of skill will recognize that the precise sequence of the particular probes described herein can be modified to a certain degree to produce probes that are “substantially identical” to the disclosed probes, but retain the ability to specifically bind to (i.e., hybridize specifically to) the same targets or samples as the probe from which they were derived (see discussion above). Such modifications are specifically covered by reference to the individual probes described herein.

As used herein, a “test nucleic acid sample” or “test nucleic acids” refer to nucleic acids comprising sequences whose quantity or degree of representation (e.g., copy number) or sequence identity is being assayed. Similarly, “test genomic acids” or a “test genomic sample” refers to genomic nucleic acids comprising sequences whose quantity or degree of representation (e.g., copy number) or sequence identity is being assayed.

As used herein, a “reference nucleic acid sample” or “reference nucleic acids” refers to nucleic acids comprising sequences whose quantity or degree of representation (e.g., copy number) or sequence identity serves as a reference to which one or more test samples are compared and more preferably the quantity or degree of representation (e.g., copy number) or sequence identity of the reference sample is known.

The term “sample” as used herein relates to a material or mixture of materials, containing one or more components of interest. Samples include, but are not limited to, samples obtained from an organism and may be directly obtained from a source (e.g., such as a biopsy or from a tumor) or indirectly obtained e.g., after culturing and/or one or more processing steps.

The term “genome” refers to all nucleic acid sequences (coding and non-coding) and elements present in each cell type, preferably each somatic cell type, of a subject. The term genome also applies to any naturally occurring or induced variation of these sequences that may be present in a mutant or disease variant of any cell type, including tumour cells. The terms “genomic DNA” and “genomic nucleic acid” are used herein interchangeably. They refer to nucleic acid isolated from a nucleus of one or more cells, and include nucleic acid derived from (i.e., isolated from, amplified from, cloned from as well as synthetic versions of) genomic DNA. For example, the human genome consists of approximately 3.0×10⁹ base pairs of DNA organised into distinct chromosomes. The genome of a normal diploid somatic human cell consists of 22 pairs of autosomes (chromosomes 1 to 22) and either chromosomes X and Y (males) or a pair of chromosome Xs (female) for a total of 46 chromosomes. A genome of a cancer cell may contain variable numbers of each chromosome in addition to deletions, rearrangements and amplification of any subchromosomal region or DNA sequence.

As used herein, the term “genomic locus” or “genomic region” refer to a defined portion of a genome. Likewise the terms “chromosomal locus” and “chromosomal region” refer to a defined portion of a chromosome. For practical purposes the terms “genomic locus”, “genomic region”, “chromosomal region” and “chromosomal locus” are used interchangeably herein. In the methods of the invention, each nucleic acid probe immobilised to a discrete spot on an array has a sequence that is specific to (or characteristic of) a particular genomic region. In an array-based comparative genomic hybridisation experiment, the ratio of intensity of two differentially labelled test and reference samples at a given spot on the array reflects the genome copy number ratio of the two samples at a particular genomic region.

If a surface-bound polynucleotide or probe “corresponds to” a genomic region, the polynucleotide usually contains a sequence of nucleic acids that is unique to that genomic region. Accordingly, a surface-bound polynucleotide that corresponds to a particular genomic region usually specifically hybridizes to a labelled nucleic acid made from that genomic region, relative to labelled nucleic acids made from other genomic regions.

“CGH” or “Comparative Genomic Hybridisation” refers generally to techniques for identification of chromosomal alterations (such as in cancer cells, for example). Using CGH, ratios between tumour or test sample and normal or reference sample enable the detection of chromosomal amplifications and deletions of regions.

The terms “tumour” or “cancer” in an animal (e.g., a human) refers to the presence of cells possessing characteristics such as atypical growth or morphology, including uncontrolled proliferation, immortality, metastatic potential, rapid growth and proliferation rate, and certain characteristic morphological features. Often, cancer cells will be in the form of a tumour, but such cells may also exist in isolation from one another within an animal. “Tumour” includes both benign and malignant neoplasms.

As used herein, “BRCA1-associated tumour” means a tumour having cells containing a mutation of the BRCA1 locus.

As used herein, “non BRCA1/2 HBOC tumours” refer to tumours in a group of patients with a high risk for BRCA1-associated breast cancer (patients from Hereditary Breast and Ovarian Cancer families) but with a negative screen result for BRCA1 and BRCA2 mutation. Such patients are from a family, which include at least two breast cancer cases and one ovarian cancer; these families are referred to as HBOC families (Hereditary Breast and Ovarian Cancer).

### DETAILED DESCRIPTION OF THE INVENTION

The present invention is based in part on the discovery that certain chromosomal copy number aberrations (CNA) in tumour cells allow to distinguish between BRCA1-associated tumours and sporadic tumours. These aberrations in chromosomal copy number comprise a set of at least 10 chromosomal regions 1p21-34, 3p21-31 (which is herein understood to mean 3p21, more preferably 3p21.1-21.31), 3q22-27, 5q13-15, 5q21-23, 6p22-23, 10p14, 12q21-23, 13q32-33 (which is herein understood to mean 13q31-33), 14q22-24 (Table 1) and some smaller regions as indicated by a list of BAC clones (Table 2). Methods wherein the copy number of at least a subset of these genomic regions is determined are useful for diagnosis and/or prognosis of breast cancer, as well as ovarium cancer and other types of tumours.

Genomic instability is a hallmark of solid tumours, and virtually no solid tumour exists that does not show some alterations of the genome. In some cases these chromosomal abnormalities are characteristic for the specific type of tumour and may thus serve as a marker for differentiation between tumour-types.

In a first aspect therefore, the present invention relates to a method for classifying a sample of cell as comprising cells from a BRCA1-associated tumour or from a sporadic tumour. The method comprises detecting the number of copies per cell in genomic DNA in the sample at least three genomic locations selected from the group consisting of 1p21-34, 3p21, 3q22-27, 5q13-15, 5q21-23, 6p22-23, 10p14, 12q21-23, 13q31-33, and 14q22-24. Preferably in the method, an increase in the number of copies per cell of DNA in genomic locations selected from the group consisting of 1p21-34, 3q22-27, 6p22-23, 10p14, and 13q31-33, and/or a decrease in the number of copies per cell of DNA in genomic locations selected from the group consisting of 3p21, 5q13-15, 5q21-23, 12q21-23, and 14q22-24, compared to the number of copies per cell in non-cancer cells, classifies the cell sample as from a BRCA1-associated tumour.

These locations may be detected individually, or in combination. Thus, for example, in some embodiments, 3, 4, 5, 6, 7, 8, 9, or 10 of the above-listed chromosomal locations may be detected. Most preferably all 10 of the above-listed chromosomal locations forementioned are detected (as are also listed in Table 1). In a preferred method of the invention the detected genomic locations are selected from the group consisting of at least 5q13-15, 3q22-27, 13q31-33, 12q21-23, 10p14, 3p21, 14q22-24, 6p22-23, and 5q21-23. In a more preferred method of the invention the detected genomic locations are selected from the group consisting of at least 5q13-15, 3q22-27, 13q31-33, 12q21-23, 10p14, 3p21, 14q22-24 and 6p22-23. In a further more preferred method of the invention the detected genomic locations are selected from the group consisting of at least 5q13-15, 3q22-27, 13q31-33, 12q21-23, 10p14, 3p21 and 14q22-24. In yet a further preferred method of the invention the detected genomic locations are selected from the group consisting of at least 5q13-15, 3q22-27, 13q31-33, 12q21-23, 10p14 and 3p21. In again a further preferred method of the invention the detected genomic locations are selected from the group consisting of at least 5q13-15, 3q22-27, 13q31-33, 12q21-23 and 10p14. In still a further preferred method of the invention the detected genomic locations are selected from the group consisting of at least 5q13-15, 3q22-27, 13q31-33 and 12q21-23. In the most preferred method of the invention the detected genomic locations are selected from the group consisting of at least 5q13-15, 3q22-27 and 13q31-33.

The methods of the invention may further comprise detecting the number of copies per cell of genomic DNA in the cell sample at least one or two genomic locations selected from 5q13-15, 3q22-27 and 13q31-33, wherein a decrease in the number of copies per cell of DNA in these genomic compared to the number of copies per cell in non-cancer cells, classifies the cell sample as from a sporadic tumour.

The above-listed genomic locations of interest in the present invention are bounded by BAC probes as listed in Table 1.

Single or low-copy number probes that detect DNA within the above genomic locations are particularly useful for use in the invention. A list of exemplary BAC clones that may be used to detect or generate probes to detect the various genomic locations is provided in Table 2. However, it should be understood that this list is not intended to limit the invention and other probes within the genomic locations can also be used. Also, the term “probe” should be understood in its broadest sense to include any nucleic acid molecule that by hybridisation with a complementary sequence in the given genomic location is capable of detecting this location. Cytogenetic banding or chromosome banding is a well-known technique to the skilled person, and e.g. Cheung et al. (2001) Nature 409:953-958; Furey and Haussler (2003) Human Molecular Genetics 12:1037-1044; and Speicher and Carter (2005) Nature Genetics 6:782-792 describe how the chromosome banding is mapped on the genome. Probe molecules for use in the methods of the invention thus range from synthetic oligonucleotide probes and/or (amplification) primers to artcificial chromosomes of more than 1 Mb, depending of the particular technique that is used for determination of copy number as described below. Probes useful in the methods described here are available from a number of sources. For instance, P1 clones are available from the DuPont P1 library (Shepard, et al., Proc. Natl. Acad. Sci. USA, 92: 2629 (1994), and available commercially from Genome Systems. Various libraries spanning entire chromosomes are also available commercially (Clonetech, South San Francisco, Calif.), or from the Los Alamos National Laboratory. The present inventors used the human 3600 BAC/PAC genomic clone set, covering the full human genome at 1 Mb spacing as may be obtained from the Welcome Trust Sanger Institute (http://www.sanger.ac.uk/). Information on this clone set can be obtained at the BAC/PAC Resources Center Web Site (http://bacpac.chori.org). Preferred probes for use in the methods of the invention comprise at least 10, 12, 15, 18, 20, 22, 30, 50 or 100 contiguous nucleotides of a (human genomic) sequence that is present in a BAC clone listed in Tables 1 and 2. More preferred nucleic acid probes comprise a sequence that is unique in the genome, preferably the human genome.

Techniques for the preparation and manipulation of nucleic acid probes are well-known in the art (see, for example, Sambrook and Russell (2001) “Molecular Cloning: A Laboratory Manual (3^{rd} edition), Cold Spring Harbor Laboratory, Cold Spring Harbor Laboratory Press, New York; P. Tijssen “Hybridisation with Nucleic Acid Probes-Laboratory Techniques in Biochemistry and Molecular Biology (Parts I and II)”, 1993, Elsevier Science; “PCR Strategies”, 1995, M. A. Innis (Ed.), Academic Press: New York, N.Y.; and “Short Protocols in Molecular Biology”, 2002, F. M. Ausubel (Ed.), 5^{th} Ed., John Wiley & Sons). Nucleic acid probes may be obtained and manipulated by cloning into various vehicles. They may be screened and re-cloned or amplified from any source of genomic DNA. Nucleic acid probes may be derived from genomic clones including mammalian and human artificial chromosomes (MACs and HACs, respectively, which can contain inserts from about 5 to 400 kilobases (kb)), satellite artificial chromosomes or satellite DNA-based artificial chromosomes (SATACs), yeast artificial chromosomes (YACs; 0.2-1 Mb in size), bacterial artificial chromosomes (BACs; up to 300 kb); P1 artificial chromosomes (PACs; about 70-100 kb) and the like. MACs and HACs have been described (see e.g. W. Roush, Science, 1997, 276: 38-39; M. A. Rosenfeld, Nat. Genet. 1997, 15: 333-335; F. Ascenzioni et al., Cancer Lett. 1997, 118: 135-142; Y Kuroiwa et al., Nat. Biotechnol. 2000, 18: 1086-1090; J. E. Meija et al., Am. J. Hum. Genet. 2001, 69: 315-326; and C. Auriche et al., EMBO Rep. 2001, 2: 102-107). SATACs can be produced by induced de novo chromosome formation in cells of different mammalian species (see e.g. P. E. Warburton and D. Kiplin, Nature, 1997, 386: 553-555; E. Csonka et al., J. Cell. Sci. 2000, 113: 3207-3216; and G. Hadlaczky, Curr. Opin. Mol. Ther. 2001, 3: 125-132). Nucleic acid probes may alternatively be derived from YACs, which have been used for many years for the stable propagation of genomic fragments of up to one million base pairs in size (see e.g J. M. Feingold et al., Proc. Natl. Acad. Sci. USA, 1990, 87:8637-8641; G. Adam et al., Plant J., 1997, 11: 1349-1358; R. M. Tucker and D. T. Burke, Gene, 1997, 199: 25-30; and M. Zeschnigk et al., Nucleic Acids Res., 1999, 27: E30). BACs may also be used to produce nucleic acid probes for use in the practice of the present invention. BACs, which are based on the E. coli F factor plasmid system, offer the advantage of being easy to manipulate and purify in microgram quantities (see e.g. S. Asakawa et al., Gene, 1997, 191: 69-79; and Y. Cao et al., Genome Res. 1999, 9: 763-774). PACs are bacteriophage P1-derived vectors (see, for example, P. A. Ioannou et al., Nature Genet., 1994, 6: 84-89; J. Boren et al., Genome Res. 1996, 6: 1123-1130; H. G. Nothwang et al., Genomics, 1997, 41: 370-378; L. H. Reid et al., Genomics, 1997, 43: 366-375; and P. Y. Woon et al., Genomics, 1998, 50: 306-316). Nucleic acid probes may also be obtained and manipulated by cloning into other cloning vehicles such as, for example, recombinant viruses, cosmids, or plasmids. Alternatively, nucleic acid sequences used as array-immobilised nucleic acid probes may be synthesised in vitro by chemical techniques well-known in the art. These methods have been described (see e.g. Nucleic Acids Res. 1997, 25: 3440-3444; M. J. Blommers et al., Biochemistry, 1994, 33: 7886-7896; and K. Frenkel et al., Free Radic. Biol. Med. 1995, 19: 373-380). An alternative to custom arraying of nucleic acid probes is to rely on commercially available arrays and micro-arrays. Such arrays have been developed, for example, by Vysis Corporation (Downers Grove, Ill.), Spectral Genomics Inc. (Houston, Tex.), and Affymetrix Inc. (Santa Clara, Calif.).

In a preferred embodiment of the method of the invention, detection of numbers of copies per cell in genomic DNA is carried out quantitatively or semi-quantitatively. It is not necessary to determine the exact copy number of the genomic regions, as detection of an aberration from the copy number in non-cancer cells, i.e. gain or loss of nucleic acid material, is sufficient. Thus, it is understood that detection of copy number includes estimation of copy numbers. Therefore, a semi-quantitative or a relative measure usually suffices. In addition, quantitative techniques may be used to determine the copy number per cell. The skilled person knows both quantitative and semi-quantitative techniques to determine copy number, e.g. semi-quantitative PCR analysis or quantitative real-time PCR.

Polymerase Chain Reaction (PCR) per se is not a quantitative technique however PCR-based methods have been developed that are quantitative or semi-quantitative in that they give a reasonable estimate of origical copy numbers within certain limits. Examples are quantitative PCR, preferably quantitative real-time PCR (known as RT-PCR, RQ-PCR, QRT-PCR or RTQ-PCR). In addition, many techniques give estimates of relative copy numbers as calculated relative to a reference, e.g. many array techniques. Absolute copy number estimates may be obtained by in situ hybridization techniques (ISH), e.g. fluorescence in situ hybridization (FISH) or chromogenic in situ hybridization (CISH) techniques. Hereafter, non-limiting examples are given of techniques that may be used for the analysis of copy numbers.

Techniques that permit the analysis of copy numbers of individual genomic locations are well known in the art. For example, fluorescence in-situ hybridization (FISH) can be used to study copy numbers of individual genetic loci or particular regions on a chromosome (Pinkel et al., Proc. Natl. Acad. Sci. U.S.A. 85, 9138-42 (1988)). Comparative genomic hybridization (CGH) (Kallioniemi et al. Science 258, 818-21 (1992)) may also be used (Houldsworth et al. Am J Pathol 145, 1253-60 (1994)) to probe for copy number changes of chromosomal regions.

Copy number of genomic locations may also be determined using quantitative PCR such as real-time PCR (see, e.g., Suzuki et al., Cancer Res. 60:5405-9 (2000)). For example, quantitative microsatellite analysis (QUMA) can be performed for rapid measurement of relative DNA sequence copy number. In QUMA, the copy number of a test locus relative to a pooled reference is assessed using quantitative, real-time PCR amplification of loci carrying simple sequence repeats. Use of simple sequence repeats is advantageous because of the large numbers that are mapped precisely. Additional protocols for quantitative PCR are provided in Innis et al. (1990) PCR Protocols, A Guide to Methods and Applications, Academic Press, Inc. N.Y.). Other semi-quantitative methods to determine specific DNA copy numbers are Multiplex Ligation-dependent Probe Amplification (MLPA) (Schouten et al. (2002) Nucleic Acids Res 30(12):e57; Sellner and Taylor (2004) Human Mutation 23(5):413-419) and Multiplex Amplification and Probe Hybridization (MAPH) (Sellner and Taylor (2004) supra).

However, preferably in the methods of the invention copy numbers of genomic locations are determined by hybridizations that are performed on a solid support. For example, probes that selectively hybridize to specific chromosomal regions can be spotted onto a surface. Conveniently, the spots are placed in an ordered pattern, or array, and the placement of the probes on the array is recorded to facilitate later correlation of results. The nucleic acid samples are then hybridized to the array. Thus, in the methods of the invention, copy numbers of genomic locations are preferably analysed in an array-based approach, e.g. using comparative genomic hybridisation. Any of a variety of arrays may be used in the practice of the present invention. Investigators can either rely on commercially available arrays or generate their own. Methods of making and using arrays are well known in the art (see, for example, S. Kern and G. M., Hampton, Biotechniques, 1997, 23:120-124; M. Schummer et al., Biotechniques, 1997, 23:1087-1092; S. Solinas-Toldo et al., Genes, Chromosomes & Cancer, 1997, 20: 399-407; M. Johnston, Curr. Biol. 1998, 8: R171-R174; D. D. Bowtell, Nature Gen. 1999, Supp. 21:25-32; S. J. Watson and H. Akil, Biol Psychiatry. 1999, 45: 533-543; W. M. Freeman et al., Biotechniques. 2000, 29: 1042-1046 and 1048-1055; D. J. Lockhart and E. A. Winzeler, Nature, 2000, 405: 827-836; M. Cuzin, Transfus. Clin. Biol. 2001, 8:291-296; P. P. Zarrinkar et al., Genome Res. 2001, 11: 1256-1261; M. Gabig and G. Wegrzyn, Acta Biochim. Pol. 2001, 48: 615-622; and V. G. Cheung et al., Nature, 2001, 40: 953-958; see also, for example, U.S. Pat. Nos. 5,143,854; 5,434,049; 5,556,752; 5,632,957; 5,700,637; 5,744,305; 5,770,456; 5,800,992; 5,807,522; 5,830,645; 5,856,174; 5,959,098; 5,965,452; 6,013,440; 6,022,963; 6,045,996; 6,048,695; 6,054,270; 6,258,606; 6,261,776; 6,277,489; 6,277,628; 6,365,349; 6,387,626; 6,458,584; 6,503,711; 6,516,276; 6,521,465; 6,558,907; 6,562,565; 6,576,424; 6,587,579; 6,589,726; 6,594,432; 6,599,693; 6,600,031; and 6,613,893). Arrays comprise a plurality of nucleic acid probes immobilised to discrete spots (i.e., defined locations or assigned positions) on a substrate surface. Substrate surfaces for use in the present invention can be made of any of a variety of rigid, semi-rigid or flexible materials that allow direct or indirect attachment (i.e., immobilisation) of nucleic acid probes to the substrate surface. Suitable materials include, but are not limited to: cellulose (see, for example, U.S. Pat. No. 5,068,269), cellulose acetate (see, for example, U.S. Pat. No. 6,048,457), nitrocellulose, glass (see, for example, U.S. Pat. No. 5,843,767), quartz or other crystalline substrates such as gallium arsenide, silicones (see, for example, U.S. Pat. No. 6,096,817), various plastics and plastic copolymers (see, for example, U.S. Pat. Nos. 4,355,153; 4,652,613; and 6,024,872), various membranes and gels (see, for example, U.S. Pat. No. 5,795,557), and paramagnetic or supramagnetic microparticles (see, for example, U.S. Pat. No. 5,939,261). When fluorescence is to be detected, arrays comprising cyclo-olefin polymers may preferably be used (see, for example, U.S. Pat. No. 6,063,338). The presence of reactive functional chemical groups (such as, for example, hydroxyl, carboxyl, amino groups and the like) on the material can be exploited to directly or indirectly attach nucleic acid probes to the substrate surface. Methods for immobilizing nucleic acid probes to substrate surfaces to form an array are well-known in the art.

More than one copy of each nucleic acid probe may be spotted on the array (for example, in duplicate or in triplicate). This arrangement may, for example, allow assessment of the reproducibility of the results obtained (see below). Related nucleic acid probes may also be grouped in probe elements on an array. For example, a probe element may include a plurality of related nucleic acid probes of different lengths but comprising substantially the same sequence. Alternatively, a probe element may include a plurality of related nucleic acid probes that are fragments of different lengths resulting from digestion of more than one copy of a cloned piece of DNA. An array may contain a plurality of probe elements. Probe elements on an array may be arranged on the substrate surface at different densities. Array-immobilised nucleic acid probes may be nucleic acids that contain sequences from genes (e.g., from a genomic library), including, for example, sequences that collectively cover a substantially complete genome or a subset of a genome. The sequences of the nucleic acid probes are those for which comparative copy number information is desired. For example, to obtain DNA sequence copy number information across an entire genome, an array comprising nucleic acid probes covering a whole genome or a substantially complete genome is used. However, in preferred embodiments of the method of the present invention the relevant genomic locations have already been established and there is no need for genome-wide experiments. In such instances the array may contain specific nucleic acid sequences that originate from a discrete set of genes or genomic locations as indicated above and whose copy number in association with the type of tumour is to be tested. Additionally, the array may comprise nucleic acid sequences as positive or negative controls (i.e., the nucleic acid sequences may be derived from karyotypically normal genomes).

Alternatively, the samples can be placed in separate wells or chambers and hybridized in their respective well or chambers. It is understood in the context of the invention that an array of separate wells or chambers is also comprsied within the general term “array” herein. The art has developed robotic equipment permitting the automated delivery of reagents to separate reaction chambers, including “chip” and microfluidic techniques, which allow the amount of the reagents used per reaction to be sharply reduced. Chip and microfluidic techniques are taught in, for example, U.S. Pat. No. 5,800,690, Orchid, “Running on Parallel Lines” New Scientist, Oct. 25, 1997, McCormick, et al., Anal. Chem. 69:2626-30 (1997), and Turgeon, “The Lab of the Future on CD-ROM?” Medical Laboratory Management Report. December 1997, p. 1. Automated hybridizations on chips or in a microfluidic environment are contemplated methods of practicing the invention. Although microfluidic environments are one embodiment of the invention, they are not the only defined spaces suitable for performing hybridizations in a fluid environment. Other such spaces include standard laboratory equipment, such as the wells of microtiter plates, Petri dishes, centrifuge tubes, or the like can be used.

In a preferred embodiment of the invention therefore includes analysing tumour cell samples by array-based comparative genomic hybridisation (aCGH). More specifically, certain methods of the invention comprise steps of: providing a sample of tumour DNA; analysing the tumour DNA by array-based comparative genomic hybridisation to obtain tumour genomic information; and, based on the tumour genomic information obtained, classifying the tumour as a BRCA1-related tumour or a sporadic tumour. The analysis step in the methods of the invention can be performed using any of a variety of methods, means and variations thereof for carrying out array-based comparative genomic hybridisation. Array-based CGH methods are known in the art and have been described in numerous scientific publications as well as in patents (see, for example, U.S. Pat. Nos. 5,635,351; 5,665,549; 5,721,098; 5,830,645; 5,856,097; 5,965,362; 5,976,790; 6,159,685; 6,197,501; 6,335,167; and EP 1 134 293 and EP 1 026 260; van Beers et al., Brit. J. Cancer, 2006; 20. Joosse et al., BMC Cancer. 2007, 7:43; D. Pinkel et al., Nat. Genet. 1998, 20: 207-211; J. R. Pollack et al., Nat. Genet. 1999, 23: 41-46; C. S. Cooper, Breast Cancer Res. 2001, 3: 158-175). In the practice of the present invention, these methods as well as other methods known in the art for carrying out array-based comparative genomic hybridisation may be used as described or modified such that they allow for tumour genomic information to be obtained. Tumour genomic information includes e.g. gain and loss of genetic material, chromosomal abnormalities and genome copy number changes at multiple genomic loci.

The method of the invention encompasses all kinds of tumours, however in a preferred embodiment of the method of the invention, a BRCA1-related tumour or a sporadic tumour is a breast tumour or an ovarian tumour. Most preferably a BRCA1-related tumour or a sporadic tumour is a breast tumour.

Test and reference nucleic acid samples for use in the methods of the present invention may be isolated from a biological sample comprising tumour or reference cells by any suitable method of DNA isolation or extraction. Methods of DNA extraction are well known in the art. A classical DNA isolation protocol is based on extraction using organic solvents such as a mixture of phenol and chloroform, followed by precipitation with ethanol (see e.g. Sambrook and Russell, 2001, supra). Other methods include: salting out DNA extraction, the trimethylammonium bromide salts DNA extraction method and the guanidinium thiocyanate DNA extraction method. There are also numerous different and versatile kits that can be used to extract DNA from bodily fluids and that are commercially available from, for example, BD Biosciences Clontech (Palo Alto, Calif.), Epicentre Technologies (Madison, Wis.), Gentra Systems, Inc. (Minneapolis, Minn.), MicroProbe Corp. (Bothell, Wash.), Organon Teknika (Durham, N.C.), and Qiagen Inc. (Valencia, Calif.). User Guides that describe in great detail the protocol to be followed are usually included in all these kits. Sensitivity, processing time and cost may be different from one kit to another. One of ordinary skill in the art can easily select the kit(s) most appropriate for a particular situation.

In the methods of the invention, the reference sample preferably is a nucleic acid sample that is representative for the normal (i.e. non-breast tumour/non-cancer cell) copy numbers of the complement of the genomic regions that are tested pool in the method in question. The reference may e.g. be derived from a genomic sample from a normal and/or healthy individual or from a pool of such individuals. Preferably the reference nucleic acid sample is from female individuals. It is also preferred that the reference nucleic acid sample does not comprise tumour DNA. A preferred reference nucleic acid sample consists of pooled genomic DNAs isolated from a tissue sample (e.g. lymphocytes) from a number (e.g. at least 4-10) of apparently healthy women. In another preferred embodiment, the reference nucleic acid sample may comprise an artificially-generated population of nucleic acids designed to approximate the level of nucleic acid sequences derived from each genomic region, or fragments thereof, of which the copy number is determined in the tumour samples. In yet another embodiment, the reference nucleic acid sample may be derived from normal cell lines or cell line samples.

In the methods of the invention the extracted test and/or reference nucleic acids may be labelled with a detectable agent or moiety before being analysed by hybridisation. Preferably, the detectable agent is selected such that it generates a signal which can be measured and whose intensity is related (e.g., proportional) to the amount of labelled nucleic acids present in the sample being analysed. In array-based hybridisation methods of the invention, the detectable agent is also preferably selected such that is generates a localised signal, thereby allowing resolution of the signal from each spot on the array.

Methods for labelling nucleic acid fragments are well-known in the art. For a review of labelling protocols, label detection techniques and recent developments in the field, see, for example, L. J. Kricka, Ann Clin. Biochem. 2002, 39: 114-129; R. P. van Gijlswijk et al., Expert Rev. Mol. Diagn. 2001, 1: 81-91; and S. Joos et al., J. Biotechnol. 1994, 35: 135-153. Standard nucleic acid labelling methods include: incorporation of radioactive agents, direct attachment of fluorescent dyes or of enzymes, chemical modifications of nucleic acid fragments making them detectable immunochemically or by other affinity reactions, and enzyme-mediated labelling methods, such as random priming, nick translation, PCR and tailing with terminal transferase. A preferred more recently developed nucleic acid labelling systems includes ULS (Universal Linkage System), which is based on the reaction of monoreactive cisplatin derivatives with the N7 position of guanine moieties in DNA (see, for example, R. J. Heetebrij et al., Cytogenet. Cell. Genet. 1999, 87: 47-52). Other suitable labelling systems include e.g. psoralen-biotin, photoreactive azido derivatives, and DNA alkylating agents.

Any of a wide variety of detectable agents can be used in the practice of the present invention. Suitable detectable agents include, but are not limited to: various ligands, radionuclides (such as for example, ³²P_{,} ³⁵S, ³H, ¹⁴C, ¹²⁵I, ¹³¹I, and the like); fluorescent dyes (for specific exemplary fluorescent dyes, see below); chemiluminescent agents (such as, for example, acridinium esters, stabilised dioxetanes and the like); microparticles (such as, for example, quantum dots, nanocrystals, phosphors and the like); enzymes (such as, for example, those used in an ELISA, i.e., horseradish peroxidase, beta-galactosidase, luciferase, alkaline phosphatase); colorimetric labels (such as, for example, dyes, colloidal gold and the like); magnetic labels (such as, for example, Dynabeads™); and biotin, dioxigenin or other haptens and proteins for which antisera or monoclonal antibodies are available.

In particularly preferred embodiments, the test and/or reference nucleic acids to be analysed by hybridisation is fluorescently labelled. Suitable fluorescent dyes for use in the present invention include e.g. Cy-3, Cy-5, Texas red, FITC, Spectrum Red, Spectrum Green, phycoerythrin, rhodamine, fluorescein, and equivalents, analogues or derivatives thereof. Favorable properties of fluorescent labelling agents to be used in the practice of the invention include high molar absorption coefficient, high fluorescence quantum yield, and photostability. Preferred labelling fluorophores exhibit absorption and emission wavelengths in the visible (i.e., between 400 and 750 nm) rather than in the ultraviolet range of the spectrum (i.e., lower than 400 nm). Preferred fluorescent dyes include Cy-3 and Cy-5 (i.e., 3- and 5-N,N′-diethyltetramethylindo-dicarbocyanine, respectively). Cy-3 and Cy-5 also present the advantage of forming a matched pair of fluorescent labels that are compatible with most fluorescence detection systems for array-based instruments (see below). Another preferred matched pair of fluorescent dyes comprises Spectrum Red and Spectrum Green. The term “differentially labelled” is used to specify that two samples of nucleic acid segments are labelled with a first detectable agent and a second detectable agent that produce distinguishable signals, whereby e.g. the first sample is the test sample and the second sample is the reference sample. Detectable agents that produce distinguishable signals include matched pairs of fluorescent dyes. Matched pairs of fluorescent dyes are known in the art and include, for example, rhodamine and fluorescein, Cy-3™ and Cy-5™, and Spectrum Red™ and Spectrum Green™.

Hybridization and wash protocols suitable for use with the methods of the invention are described, e.g., in Sambrook and Russell, 2001, supra, P. Tijssen “Hybridisation with Nucleic Acid Probes-Laboratory Techniques in Biochemistry and Molecular Biology (Part II)”, Elsevier Science, 1993; and “Nucleic Acid Hybridisation”, M. L. M. Anderson (Ed.), 1999, Springer Verlag: New York, N.Y. Preferred the hybridization protocols for CGH are those of Pinkel et al. (1998) Nature Genetics 20:207-211 or of Kallioniemi (1992) Proc. Natl. Acad Sci USA 89:5321-5325 (1992). Methods of optimizing hybridization conditions are well known to those of skill in the art (see, e.g., Tijssen, 1993, supra). In order to create competitive hybridisation conditions, the array may be contacted simultaneously with the (differentially) labelled nucleic acid fragments of the test and reference samples. This may be done by, for example, mixing the test and reference samples to form a hybridisation mixture and contacting the array with the mixture.

The specificity of hybridisation may further be enhanced by inhibiting repetitive sequences. In certain preferred embodiments, repetitive sequences sequences (e.g., Alu, L1 and satellite sequences, MRE sequences and simple homo- or oligo-nucleotide tracts) present in the nucleic acid fragments are removed or their hybridisation capacity is disabled. Removing repetitive sequences from a mixture or disabling their hybridisation capacity can be accomplished using any of a variety of methods well-known to those skilled in the art. These methods include, but are not limited to, removing repetitive sequences by hybridisation to specific nucleic acid sequences immobilised to a solid support (see e.g. O. Brison et al., Mol. Cell. Biol. 1982, 2: 578-587); suppressing the production of repetitive sequences by PCR amplification using adequate PCR primers; inhibiting the hybridisation capacity of highly repeated sequences by self-reassociation (see e.g R. J. Britten et al., Methods of Enzymology, 1974, 29: 363-418); or removing repetitive sequences using hydroxyapatite (which is commercially available, for example, from Bio-Rad Laboratories, Richmond, Va.). Preferably, the hybridisation capacity of highly repeated sequences is competitively inhibited by including, in the hybridisation mixture, unlabelled blocking nucleic acids. The unlabelled blocking nucleic acids, which are mixed to the test and reference samples before the contacting step, act as a competitor and prevent the labelled repetitive sequences from binding to the highly repetitive sequences of the nucleic acid probes, thus decreasing hybridisation background. In certain preferred embodiments, the unlabelled blocking nucleic acids are Human Cot-1 DNA. Human Cot-1 DNA is commercially available, for example, from Gibco/BRL Life Technologies (Gaithersburg, Md.).

In another aspect the invention therefore relates to a set of at least three nucleic acid probes for use in the above described methods of the invention. In the set preferably each probe specifically hybridises to a different genomic location selected from the group consisting of 5q13-15, 3q22-27, 13q31-33, 12q21-23, 10p14, 3p21, 14q22-24, 6p22-23, and 5q21-23. More preferably in the set each probe specifically hybridises to a different genomic location selected from the group consisting of 5q13-15, 3q22-27, 13q31-33, 12q21-23, 10p14, 3p21, 14q22-24 and 6p22-23. Further preferred in the set each probe specifically hybridises to a different genomic location selected from the group consisting of 5q13-15, 3q22-27, 13q31-33, 12q21-23, 10p14, 3p21 and 14q22-24. Yet further preferred in the set each probe specifically hybridises to a different genomic location selected from the group consisting of 5q13-15, 3q22-27, 13q31-33, 12q21-23, 10p14 and 3p21. Again further preferred in the set each probe specifically hybridises to a different genomic location selected from the group consisting of 5q13-15, 3q22-27, 13q31-33, 12q21-23 and 10p14. Still further preferred in the set each probe specifically hybridises to a different genomic location selected from the group consisting of 5q13-15, 3q22-27, 13q31-33 and 12q21-23. Most preferably, in the set each probe specifically hybridises to a different genomic location selected from the group consisting of 5q13-15, 3q22-27 and 13q31-33. In these sets, the nucleic acid probes for detection of genomic locations are as defined above and/or may be obtained in methods as described above.

In another aspect the invention relates to a BAC clone, which BAC clone is selected from the group of BAC clones as listed in Table 1 or Table 2. Preferably the invention relates to a set of at least three BAC clones selected from the group of BAC clones as listed in Table 1 or Table 2. More preferably, the set comprises at least 4, 5, 6, 7, 8, 9, 10, 15, 20, 30, 50, 60, 80 or 100 BAC clones selected from the group of BAC clones as listed in Table 1 or Table 2. Even more preferably, the set of BAC clones represent at least three, more preferably at least 4, 5, 6, 7, 8, 9 or 10 of the above-listed genomic locations. Most preferably, the set of BAC clones represent all 10 of the above-listed chromosomal locations (as are also listed in Table 1).

In yet another aspect the invention pertains to an array comprising a set of at least three nucleic acid probes for use in the above described methods of the invention as defined above. More preferably the array comprises distinct nucleic acid probes and/or distinct BAC clones that specifically hybridise to at least 4, 5, 6, 7, 8, 9, 10 of the above-listed genomic locations. More preferably the array comprises nucleic acid probes that comprises a sequence that is unique in the above-listed genomic locations. Most preferably the array comprises distinct probes and/or distinct BAC clones for all 10 of the above-listed chromosomal locations (as are are also listed in Table 1). It is understood herein that an array that comprises distinct nucleic acid probes and/or distinct BAC clones that specifically hybridise to at least three genomic locations is an array that allows to individually analyse at least three (different) genomic locations. Thus, preferably a set of nucleic acid probes and/or BAC clonese is arranged on the array in a positionally-addressable manner. An array is herein understood as any solid support onto which the probes are immobilised, whereby preferably the probes and or BAC clones are immobilised onto the solid support in a positionally-addressable manner. Preferably, the distinct BAC clones that are comprised on the array are selected from the group of BAC clones as listed in Table 1 or Table 2.

In a further aspect the invention relates to kits for use in the diagnostic applications described above. The kits of the invention may comprise any or all of the reagents to perform the methods described herein. In the diagnostic applications such kits may include any or all of the following: assay reagents, buffers, nucleic acids such hybridization probes and/or primers that specifically bind to at least one of the genomic locations described herein, as well as arrays comprising such nucleic acids. In addition, the kits may include instructional materials containing directions (i.e., protocols) for the practice of the methods of this invention. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

In yet a further aspect the invention relates to a diagnostic and/or prognostic method for indicating the involvement of a BRCA1 deficiency in the development of a tumour in a subject. The method preferably comprises the use of a method as defined hereinabove, a set of nucleic acid probes as defined hereinabove, an array as defined hereinabove, or a kit as defined hereinabove. The diagnostic and/or prognostic method may further be used (on its own or as an additional tool) to identify BRCA1-mutation carrying families where the BRCA1-relation is still unclear. The diagnostic and/or prognostic method may also be used to select for the individual within a high risk family for intensive DNA screening most likely carrying a mutation and/or the diagnostic and/or prognostic method may be used to guide DNA-diagnostics. Additionally, the diagnostic and/or prognostic method may be used to give indications for the significance of unclassified variants. The methods of the invention provide a reliable test for indicating the involvement of a BRCA1 deficiency in the development of individual tumours and as such support decision making in genetic counselling and clinical management, e.g. of the treatment of breast tumours.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”.

All patent and literature references cited in the present specification are hereby incorporated by reference in their entirety.

The following examples are offered for illustrative purposes only, and are not intended to limit the scope of the present invention in any way.

### DESCRIPTION OF THE FIGURES

FIG. 1. Chromosomes (X-axis) versus the percentage (Y-axis) of samples showing gain (gray, log 2 ratios>0.2) or loss (black, log 2 ratios<0.2) for BRCA1 (A) and sporadic (B) breast carcinomas.

FIG. 2. Distribution of the discriminant scores of the different tumour groups for the train and validation sets plotted in boxplots (A). BRCA1-associated tumours are plotted at the positive values and sporadic tumours at the negative values. Based on these values 95% reference intervals were set for the BRCA1-asocciated group on 1.7 and for the sporadic group on −1.0 (dotted lines). Applying the classification rule to our HBOC group, 2 tumours were classified as BRCA1-like (B, black dots).

FIG. 3. Class predictor robustness. Fifteen independent random selections of training sets resulted every time in a classification accuracy of 100%. Training sets are plotted (left) with the corresponding validation sets (right) for the BRCA1 samples at the positive values and the sporadic tumour samples at negative values. Dotted lines represent the median values for the corresponding group of sets.

FIG. 4. Complete hierarchical clustering based on the array-CGH results of the BRCA1-related and sporadic tumours accompanied with their pathological characteristics ER, PR, HER2/Neu, and TP53-status. Although there is some separation between BRCA1-associated and sporadic tumours, pathological characteristic do not reside in clear clusters.

### EXAMPLES

### 1.1 Methods and Materials

1.1.1 Patients and Sample selection

This study was performed on three breast cancer groups: 1) 28 breast tumours from patients with a verified pathogenic BRCA1 germline mutation, mean age at diagnosis of 39 years (range: 27-61); 2) 48 sporadic breast tumours with mean age at diagnosis of 45 years (range: 32-60), without family history for breast cancer, and were randomly selected from the institutes archive, however, with the same percentage of P53-negative and positive samples as the BRCA1-associated tumour group (Table 3). Both BRCA1-associated and sporadic tumour groups consisted of invasive grade II-III ductal carcinomas. 3) 48 tumours from HBOC families (at least two breast and one primary ovarian cancer), that were subjected to routine diagnostic testing (described by Van der Hout 2006) and had a negative test result for mutations in both BRCA1 and BRCA2. The mean age at diagnosis was 48 years (range: 20-61). Patient's characteristics for all three groups are described in the supplementary table patient information. All sample material was formalin-fixed, paraffin-embedded (FFPE) tissue and extracted DNA had to be of sufficient quality as described before (Van Beers 2006). All experiments involving human tissues were conducted with permission of the institutes' medical ethical advisory board.

### 1.1.2 DNA Isolation

Sample DNA was isolated from FFPE tumour tissues as follows. Ten 10 μm slices containing at least 70% tumour cells were cleared of paraffin (2×5 min xylene, 2×30s 100% ethanol, 30s 90% ethanol, 30s 70% ethanol, and rinsed with H2O), treated with 1M sodium acetate at 37° C. over night, and sections of interest (>70% tumour cells) were scraped in 200 μl buffer ATL (Qiagen, cat.no. 51304). 27 μl proteinase K (15 μg/μl, Roche, cat no 3115879001) was immediately added, the same amount at the end of the day and the beginning and the end of the next day; samples were kept shaking at 37° C. all time of digestion. The following day, 40 μl RNase A (20 μg/μl, Sigma, cat.no.RS500) was added to the sample, vortexed, and incubated for 2 minutes at room temperature. 400 μl buffer AL (Qiagen, cat.no. 51304) was added and incubated for 10 minutes at 70° C. 420 μl 100% ethanol was added and vortexed. Sample mixture was spun on a spincolumn (Qiagen, cat.no. 51304) for 1 minute at 8000 rpm. Column was washed with the following reagents sequentially and spun for 1 minute at 8000 rpm: 500 μl AW1, 500 μl AW2, and twice with 80% ethanol. The column was spun dry for 3 minutes at 14,000 rpm. Sample was eluted with 50 μl AE buffer by spinning for 1 minute at 8000 rpm.

Reference DNA was isolated from lymphocytes from six apparently healthy women and pooled. Lymphocytes were purified by adding lysis buffer (155 mM NH4Cl, 10 mM KHCO3, 1 mM EDTA) four times the blood volume, followed by centrifugation at 3000 rpm for 10 minutes at 4° C. Supernatant was removed and cell pellet re-suspended in lysis buffer five times the original blood volume. These steps were repeated until all erythrocytes were removed and the supernatant was a clear solution. 1/10 of the initial blood volume DNAzo1 (Invitrogen, cat.no. 10503-027) was added to the cell pellet and mixed by pipeting until a clear solution was left. ½ of the DNAzo1 volume 100% ethanol was added, DNA was removed from the solution, washed in 70% ethanol and dissolved in Tris-EDTA buffer. DNA was sonicated until the average length was 300-800 bp.

### 1.1.3 Quality Assay

To test the suitability of sample DNA for array-CGH, a quality PCR was performed as described before (Van Beers 2006). This multiplex PCR contains primes to produce band of 100, 200, 300, and 400 bp. Depending on the quality of the DNA, the PCR will produce the different bands. DNA from which at least the 200 by fragment can be amplified is of sufficient quality for array-CGH.

### 1.1.4 Hybridisation

As described before (Joosse 2007), hybridisations were done on micro arrays containing 3.5 k BAC/PAC derived DNA segments covering the whole genome with an average spacing of 1 Mb. The whole library was in triplicate spotted on every slide (Code Link Activated Slides, Amersham Biosciences, Prod. No. 300011 00).

### 1.1.5 Measurement Quantification

Data processing of the scanned microarray slide included signal intensity measurement in ImaGene Software followed by median pin tip (c.q. subarray) normalisation. Intensity ratios (Cy5/Cy3) were log 2 transformed and triplicate spot measurements were averaged. Chromosomal breakpoints and aberrations were calculated using CGH-segmentation (Picard 2005).

### 1.1.6 Class Prediction

To build a class predictor based on log₂ (ratios) of our CGH experiments, the shrunken centroids algorithm (Tibshirani, 2002) was used. For calculating the squared distances δₖ for each class K to the sample x*, we have applied equal priors (πₖ=1/K). As the shrinkage increases, the number of BAC clones dividing the tumour groups d′ᵢₖ decreases, thereby, also the squared distances become relatively small.

Analogy to Gaussian linear discriminant analysis was therefore not applied. The different dynamic ranges and the difference in CGH profiles between samples give a wide variability in discriminant scores. This variability affects both classes' scores and can therefore be scaled towards zero by subtracting both scores with the smallest discriminant score:

δₖ′(x*)=δₖ(x*)−arg minₖδ(x*)

The classification rule for sample x* is where δₖ′ (x*)=0. The arg max δ′ₖ(x*) is the distance to the improbable class for sample x*. We use arg max δ′ₖ (x*) here as a ‘likelihood score’ for the probable class.

The class predictor was built on 18 random selected BRCA1-mutated and 32 sporadic breast tumours, and validated on an independent set of 10 BRCA1-mutated and 16 sporadic tumours. To be able to test the HBOC tumour group, reference intervals were based on 95% of the training and validation scores. For legibility, scores for the sporadic tumour group are shown negative, BRCA1 scores positive.

### 1.1.7 Methylation Detection

Hypermethylation of BRCA1 promoter was determined by using methylation MLPA according to manufactures' protocol (MRC-Holland, ME001), with a PCR of 30 cycles. Two micro litre MLPA-PCR product was added to 9.8 μl Hi-Di formamide (AB, 4311320) and 0.2 μl ROX-500 (AB, 401734), and analysed on a 3730 DNA sequencer (AB).

### 1.1.8 Loss of Heterozygosity

LOH at the BRCA1 locus was determined using 5 markers: D17S579, D17S588, D17S1322, D17S1323, and THRAl. Primers and PCR program are described in Tables 4 and 5. One micro litre PCR product was added to 14.9 μl Hi-Di formamide and 0.1 ROX-350 (AB, 401735), and analysed using the 3730 DNA Analyzer.

### 1.1.9 GEO

Microarray data have been deposited in NCBIs Gene Expression Omnibus and are accessible through GEO Series accession number GSE9021 (BRCA1-associated tumours) and GSE9114 (sporadic tumours).

### 2. Results

In total, we have obtained array-CGH profiles of 28 BRCA1-related, 48 sporadic and 48 HBOC breast tumours. We here report the chromosomal aberrations and their locations, the differences between the tumour groups, and the discriminating power of a class predictor based on our CGH results.

### 2.1 Chromosomal Aberrations

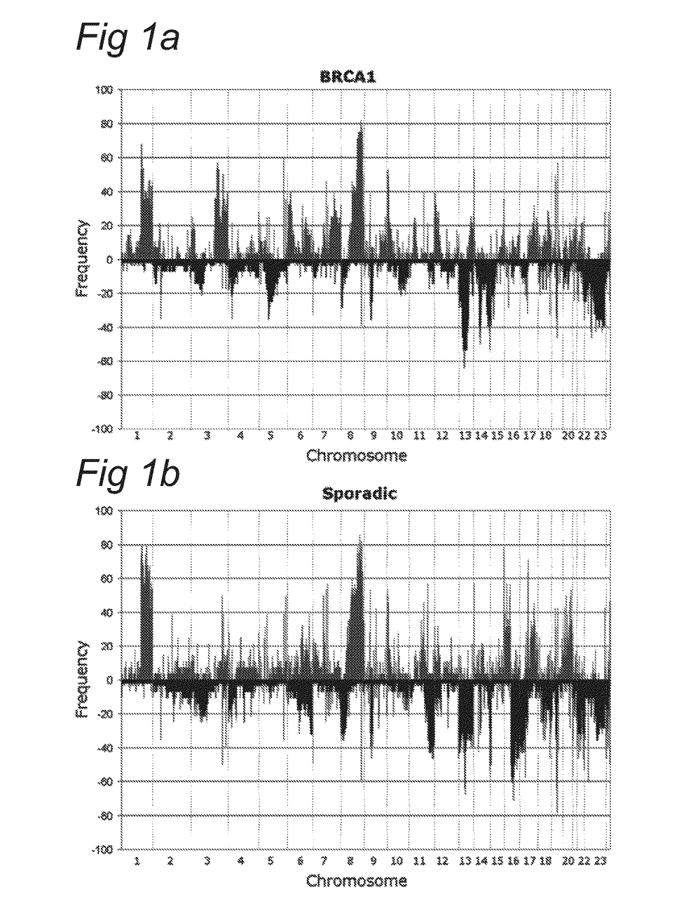

To analyse chromosomal aberrations, we determined breakpoint locations and estimated copy number levels using the CGH-segmentation algorithm (Picard 2005). Based on the estimated copy number levels, the frequency for gain and loss for all BAC clones was calculated using fixed log 2 ratio thresholds of 0.2 and −0.2 respectively. FIG. 1 depicts the frequency of gain (gray) and loss (black) of the BAC clones for the BRCA1-associated (FIG. 1A) and the sporadic (FIG. 1B) breast tumours. Location and average frequencies of the aberrations are described in detail in Table 6. In the BRCA1-associated tumours thirteen regional (>10 Mb) gains and twelve regional losses were observed in >20% of the tumours. Using the same criteria, we observed in the sporadic breast tumours gain in four chromosomal regions and six regional losses. Calculated with the t-test, twenty-one of these aberrations found in the BRCA1-related tumours were significantly different from those regions in the sporadic cases (p<0.001), seven aberrations found in the sporadic tumours were significantly different from the BRCA1-associated tumours in the same regions (p<0.001). In total, four aberrations were not significantly different between the two groups (p>0.001).

### 2.2 BRCA1 and Sporadic Breast Tumour Class Predictor

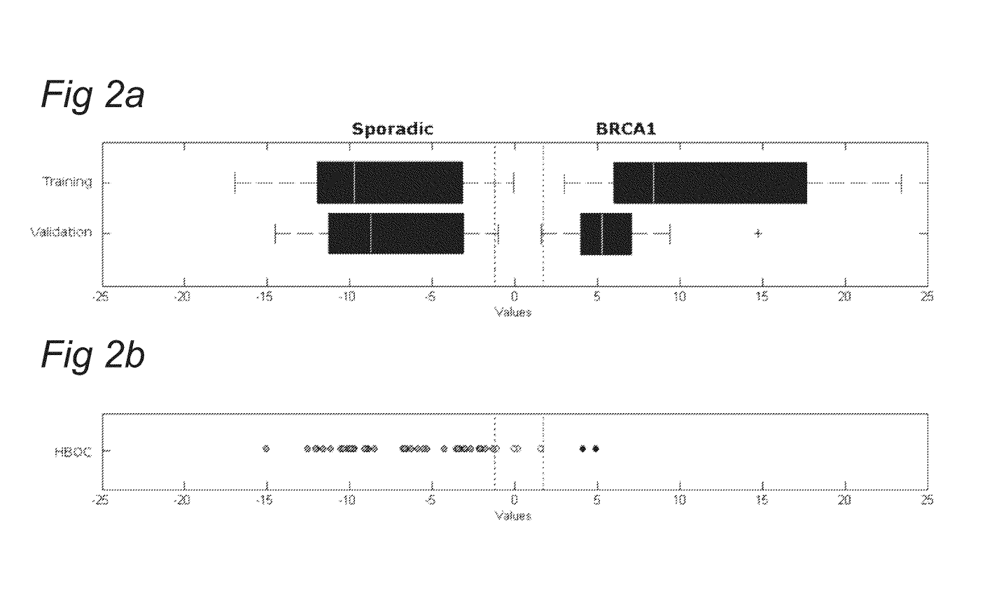

We have used nearest Shrunken Centroids (SC) as classification method to discriminate between two breast cancer types, germ line mutated BRCA1 (class 1) and sporadic tumours (class 2). Two third of the samples of each set were randomly selected, i.e. 18 BRCA1 and 32 sporadic tumours, for the SC analysis. Based on leave-one-out cross-validation (LOOCV) (supplementary data LOOCV), the analysis was performed using Δ=1.3 (Van Beers 2006, formula 5) and 191 features were selected to be discriminatory. The remaining one third of the samples were used as external validation for the class predictor. All samples of the BRCA1 group (n=10) were predicted as BRCA1-like and all sporadic samples (n=16) were classified correctly as sporadic tumours. Features that were selected as most characteristic for BRCA1 breast tumours were abundant in regions of chromosome 3q22-27 (gain), 5q12-14 (loss), 6p23-22 (gain), 10p15-14 (gain), 12p13 (gain), 12q21-23 (loss), and 13q31-34 (gain). Features that were specific for the sporadic tumour set were abundant in regions of chromosome 3q22-26 (loss) and 13q31-33 (loss). BAC clones that were selected using the SC method are listed in Tables 1 and 2. FIG. 2A depicts the distribution of the discriminant scores of the sample groups in a boxplot. This figure illustrates how well both tumour groups can be discriminated from each other.

### 2.3 Performance Validation

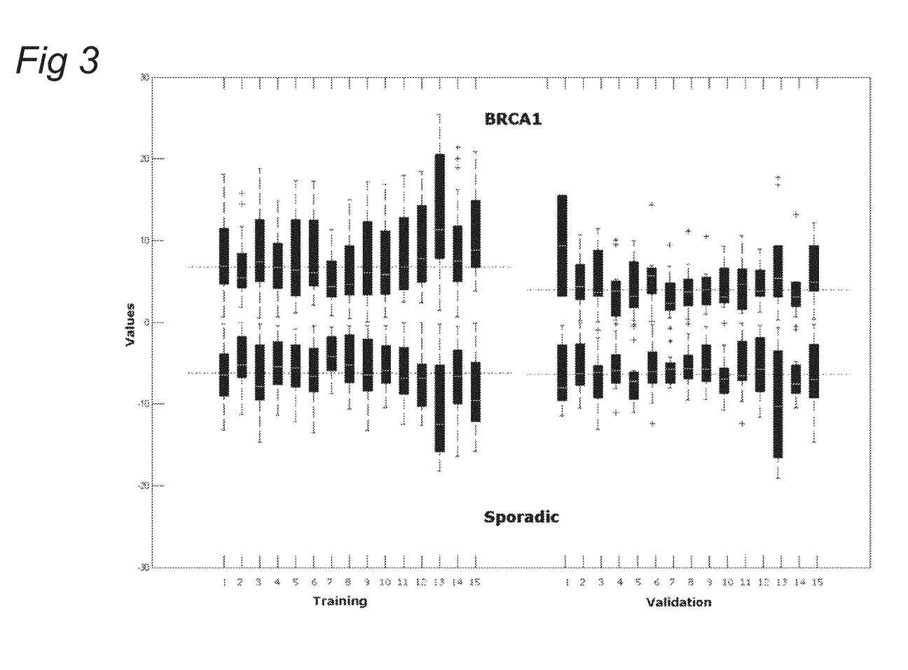

Robustness of our classification predictor was further tested by 15-fold random selections of two-third of the data set for training a class predictor and validating on the remaining one-third of the samples. All fifteen different permutations resulted in a performance of 100% as can be seen in FIG. 3.

### 2.4 Receptor and P53 Involvement

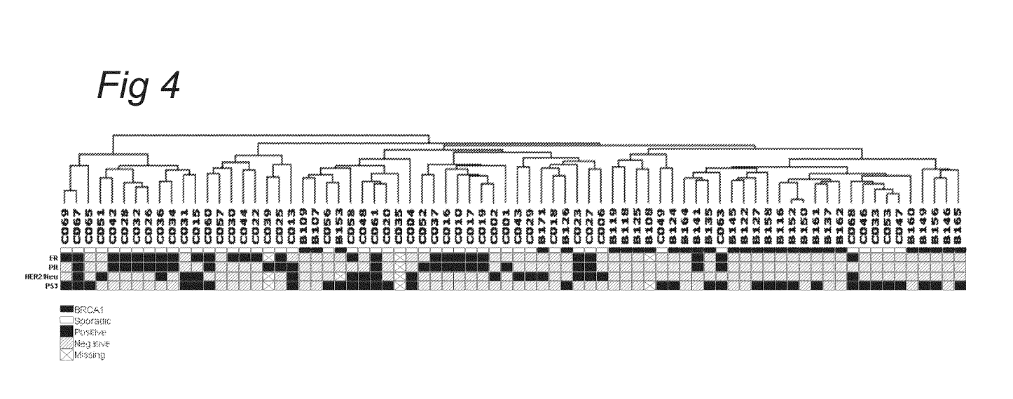

In our tumour groups, BRCA1-mutated tumours are generally ER, PR and HER2/Neu negative (also known as triple negative), and only 19% of the sporadic cases are triple-negative (supplementary table sample information). To investigate the relation of ER, PR, HER2/neu, or P53-status with chromosomal aberrations and thus the influence on our class predictor, hierarchical cluster analysis (Eisen 1998) was performed on the array CGH results of the 28 BRCA1-associated and the 48 sporadic breast tumours. Tumours sharing the same receptor or P53-status did not reside in clusters as can be seen in FIG. 4. These results indicate that there was no association with ER, PR, HER2/neu, or P53-status and our CGH results.

### 2.5 Application of the Classifier in the Clinic

Fourty-eight patients from HBOC families (at least two patients with breast carcinoma and at least one case of primary ovarian carcinoma), were selected and analysed using aCGH. Applying the class predictor, we found 2 samples to be BRCA1-like, 42 samples were predicted as sporadic cancer, and 4 samples could not be assigned with certainty to a class as they fell outside the 95% reference intervals. FIG. 2B shows the distribution of the clinical samples in comparison with the BRCA1-related and sporadic tumours used to build and validate our class predictor (FIG. 2A). Samples that appear BRCA1-like were HR015 and HR019. Information on all patients can be found in Tables 7A-7C.

To find evidence for BRCA1 involvement in the breast cancer cases that we have classified as BRCA1-like, we performed additional tests that were not included in the original diagnostic setting (Van der Hout 2006). Hypermethylation of the promoter of BRCA1 was determined for all BRCA1-associated, sporadic, and HBOC samples using MLPA-methylation (MRC-Holland, ME001). Only case HR015, which was classified as BRCA1-like, was hypermethylated at the BRCA1 promoter. Additional analyses show also hypermethylation at the BRCA1 promoter site within the ovarian tumour of the same patient, but interestingly not in her lymphocyts. Loss of Heterozygosity (LOH) of BRCA1 was observed in both the samples HR015 and HR019. As BRCA1 exon 11 is the gene's largest exon (it codes for 61% of the protein) and is approximately 3.4 kB long, sequencing is not a standard diagnostic procedure, but is screened for truncating mutations by PTT (Hogervorst 1995). We sequenced exon 11 without finding any mutations. For case HR019 no methylation of BRCA1, or unclassified variants were identified, however, loss of one BRCA1 allele was observed in the tumour.

### 3. Discussion

We show that BRCA1-associated breast tumours develop rearranged genomes with specific genomic aberrations that differ significantly from sporadic breast tumours. Based on our array-CGH data, we were able to identify the most significant differences between these two tumour groups and have built a class predictor with a sensitivity and specificity of 100% using the Shrunken Centroids method (Tibshirani 2002). Compared with the BRCA1-associated tumours, aberrations are seen less frequently in sporadic breast tumours. Many of the identified regions specific for the BRCA1-related tumours have been published (Tirkkonen 1997, Wessels 2002, Van Beers 2005, Jonsson 2005, Johannsdottir 2006) but have been poorly correlated to combined receptor and P53-status. It has been reported that BRCA1 tumours are in general ER, PR, and HER2/neu-negative (Lakhani 2002). Furthermore, it has been shown that specific genetic alterations are associated with receptor and P53-status (Loo 2004, Fridlyand 2006). The differences in chromosomal aberrations between ER-negative and positive breast carcinomas are located at 4p16, 5q23-35, 8p23-21, 10p12, 10q25, 17q11, 19q13, and 21q22 (Loo 2004). The differences in chromosomal aberrations between P53-positive and negative breast tumours are 3p, 4q, 5q, 8q, 15q, and 17q (Fridlyand 2006). To prevent possible influence of P53-status on the separation of our tumour groups, equal distributions of P53-negative and positive tumours were used to build our class predictor. Since we did not have equal numbers of ER-negative tumours in both tumour classes (since this is dominant in BRCA1-associated tumours), we will discuss here whether receptor status may have influenced our class predictor. Although the ER and P53-specific chromosomal regions could be confirmed in our CGH data (data not shown), only a small region of 5q was present in our class predictor, indicating that ER and P53-status do not strongly influence the classifier. Wessels et al. already classified BRCA1-associated and sporadic tumours using classical CGH with an accuracy of 84%, here loss in 3p and 5q and gain in 3q were identified as discriminatory aberrations. As described by Fridlyand et al., TP53-mutatand tumours show loss in chromosome 3p. This chromosomal region is part of the classifier of Wessels et al. which could have contributed to false positives. Chromosomal regions 3q and 5q are present in our current class predictor, however not 3p. This suggests that an equal distribution of P53-tumours in both tumour groups could have helped achieving a better specificity.

Performing unsupervised cluster analyses on the array-CGH results, tumours from the BRCA1-related and sporadic tumour groups sharing the same receptor or P53-status do not reside in clusters (FIG. 4). Unsupervised clustering, 100% classification performance, and the equal percentages of P53-positive tumours in both tumour classes strongly indicate the absence of P53, ER, PR, Her2neu specific chromosomal imbalances on our tumour prediction.

There are other studies reporting BRCA1-status prediction based on clinico- and pathological reviewing (Lakhani 2005, Van der Groep 2006). These studies show that the investigated protein expressions could not all be clearly related to dysfunctional BRCA1, suggesting the difficulty of pathological reviewing with the currently available markers.

Applying our classification technique to breast tumours from non-BRCA1/2 families, we identified 2 out of 48 tumours to be BRCA1-like. Because all tumours were formalin-fixed and paraffin-embedded, we could not investigate for BRCA1 RNA expression. However, further analyses on genomic DNA showed hypermethylation and LOH of the BRCA1 gene in one of these cases, strongly indicating BRCA1 dysfunction. Cancer formation due to BRCA1 mutation is generally accompanied by the loss of the wild type allele, i.e. LOH, which was also found in the second BRCA1-like HBOC tumour. However, no novel or described mutations in the BRCA1 gene could be identified in this tumour after sequencing exon 11. One explanation for finding no evidence yet for BRCA1-involvement in tumour formation could be that this tumour has sporadically arisen but does suffer of BRCA1-dysfunction (Turner 2007). This particular patient's family history was different compared with an average BRCA1-involved family (breast and ovarian cancer) and included also brain, colon cancer, and leukaemia, additionally, the tumour was ER and PR-positive which is uncommon for BRCA1-related tumours (Lakhani 2002). This unresolved BRCA1-like case has to be analysed more intensively when new techniques and knowledge are available.

These two BRCA1-like tumours were calculated to have a chance for having a BRCA1-mutation according to the Evans scoring (Evans 2004) of 20% and 11.8%, respectively, which is surprisingly low compared with the tumours with an Evans score>50%, that were not classified to be BRCA1-like. This suggests that risk prediction based on family history is not perfect; also, sporadic tumours that have dysfunctional BRCA1 (Turner 2007) can obviously not be predicted using family based models.

Some of the reasons for the variety of discriminant scores within the BRCA1-associated and the sporadic tumour groups are the technical variances between log 2 ratios; also an over estimation of tumour percentage, which can cause a suppressed tumour profile, can lead to a false classification. Therefore, we applied reverence intervals which are based on 95% of our data. Four of our tested HBOC breast carcinomas ended up outside the 95% reference intervals of our classifier. Since discriminant scores outside the 95% reference intervals become too small to be reliable, we withhold to classify these cases.

Although further validation in a large series is required, we conclude that current diagnostics does find most hereditary BRCA1-associated breast tumours. However, while we could still find BRCA1-related breast tumours, our approach may also be used as an additional tool to identify BRCA1-mutation carrying families where BRCA1-relation is still unclear. In the future, it may be possible to include this test into diagnostic routine to select for the individual within a high risk family for intensive DNA screening most likely carrying a mutation, and may be used to guide DNA-diagnostics. Additionally, it may give indications for the significance of unclassified variants (Tischkowitz submitted). Our method outperforms pathological reviewing and all other available methods on tumour material in predicting clinical samples for BRCA1-association.

### REFERENCES

1. Visser O, Coebergh J W W, van Dijck J A A M, Siesling S. Incidence of cancer in the Netherlands 1998. Tenth report of the Netherlands Cancer Registry. Utrecht (the Netherlands): Association of Comprehensive Cancer Centres; 2002. ISBN:90-72175-32-B.
2. American Cancer Society. Atlanta (GA). Breast cancer facts and figures. 2003-2004. Available from: http://www.cancer.org/.
3. Szabo C I, King M C. Population genetics of BRCA1 and BRCA2. Am J Hum Genet. 1997 May; 60(5):1013-20.
4. van der Hout A H, van den Ouweland A M, van der Luijt R B, Gille H J, Bodmer D, Bruggenwirth H, Mulder I M, van der Vlies P, Elfferich P, Huisman M T, ten Berge A M, Kromosoeto J, Jansen R P, van Zon P H, Vriesman T, Arts N, Lange M B, Oosterwijk J C, Meijers-Heijboer H, Ausems M G, Hoogerbrugge N, Verhoef S, Halley D J, Vos Y J, Hogervorst F, Ligtenberg M, Hofstra R M. A DGGE system for comprehensive mutation screening of BRCA1 and BRCA2: application in a Dutch cancer clinic setting. Hum Mutat. 2006 July; 27(7):654-66.
5. Easton D F, Ford D, Bishop D T. Breast and ovarian cancer incidence in BRCA1-mutation carriers. Breast Cancer Linkage Consortium. Am J Hum Genet. 1995; 56:265-71.
6. Ford D, Easton D F, Stratton M, et al. Genetic heterogeneity and penetrance analysis of the BRCA1 and BRCA2 genes in breast-cancer families. The Breast Cancer Linkage Consortium. Am J Hum Genet. 1998; 62:676-89.
7. van der Groep P, Bouter A, van der Zanden R, Siccama I, Menko F H, Gille J J, van Kalken C, van der Wall E, Verheijen R H, van Diest P J. Distinction between hereditary and sporadic breast cancer on the basis of clinicopathological data. J Clin Pathol. 2006 June; 59(6):611-7.
8. Antoniou A, Pharoah P D, Narod S, et al. Average risks of breast and ovarian cancer associated with BRCA1 or BRCA2 mutations detected in case series unselected for family history: a combined analysis of 22 studies. Am J Hum Genet. 2003; 72:1117-30 .
9. King M C, Marks J H, Mandell J B. Breast and ovarian cancer risks due to inherited mutations in BRCA1 and BRCA2. Science 2003; 302:643-6.
10. Tercyak K P, Peshkin B N, Brogan B M, Demarco T, Pennanen M F, Willey S C, Magnant C M, Rogers S, Isaacs C, Schwartz M D. Quality of Life After Contralateral Prophylactic Mastectomy in Newly Diagnosed High-Risk Breast Cancer Patients Who Underwent BRCA1/2 Gene Testing. J Clin Oncol. 2006 Dec. 11;
11. Cleator S, Heller W, Coombes R C. Triple-negative breast cancer: therapeutic options. Lancet Oncol. 2007 March; 8(3):235-44. Review.
12. Narod S A, Goldgar D, Cannon-Albright L, Weber B, Moslehi R, Ives E, Lenoir G, Lynch H. Risk modifiers in carriers of BRCA1 mutations. Int J Cancer. 1995 Dec. 20; 64(6):394-8.
13. Perou C M, Sorlie T, Eisen M B, van de Rijn M, Jeffrey S S, Rees C A, Pollack J R, Ross D T, Johnsen H, Akslen L A, Fluge O, Pergamenschikov A, Williams C, Zhu S X, Lonning P E, Borresen-Dale A L, Brown P O, Botstein D. Molecular portraits of human breast tumours. Nature. 2000 Aug. 17; 406(6797):747-52.
14. Hedenfalk I, Duggan D, Chen Y, Radmacher M, Bittner M, Simon R, Meltzer P, Gusterson B, Esteller M, Kallioniemi O P, Wilfond B, Borg A, Trent J, Raffeld M, Yakhini Z, Ben-Dor A, Dougherty E, Kononen J, Bubendorf L, Fehrle W, Pittaluga S, Gruvberger S, Loman N, Johannsson 0, Olsson H, Sauter G. Gene-expression profiles in hereditary breast cancer. N Engl J. Med. 2001 Feb. 22; 344(8):539-48.
15. Wessels L F, van Welsem T, Hart A A, van't Veer L J, Reinders M J, Nederlof P M. Molecular classification of breast carcinomas by comparative genomic hybridization: a specific somatic genetic profile for BRCA1 tumors. Cancer Res. 2002 Dec. 1; 62(23):7110-7.
16. Van Beers E H, van Welsem T, Wessels L F, Li Y, Oldenburg R A, Devilee P, Cornelisse C J, Verhoef S, Hogervorst F B, van't Veer L J, Nederlof P M. Comparative genomic hybridization profiles in human BRCA1 and BRCA2 breast tumors highlight differential sets of genomic aberrations. Cancer Res. 2005 Feb. 1; 65(3):822-7.
17. Jonsson G, Naylor T L, Vallon-Christersson J, Staaf J, Huang J, Ward M R, Greshock J D, Luts L, Olsson H, Rahman N, Stratton M, Ringner M, Borg A, Weber BL. Distinct genomic profiles in hereditary breast tumors identified by array-based comparative genomic hybridization. Cancer Res. 2005 Sep. 1; 65(17):7612-21.
18. Van Beers E H, Joosse S A, Ligtenberg M J, Fles R, Hogervorst F B L, Verhoef S, Nederlof P M: A multiplex PCR predictor for aCGH success of FFPE samples. Br J. Cancer. 2006 January; 94(2):333-7
19. Kang H H, Williams R, Leary J; kConFab Investigators; Ringland C, Kirk J, Ward R. Evaluation of models to predict BRCA germline mutations. Br J Cancer. 2006 Oct. 9; 95(7):914-20.
20. Joosse S A, van Beers E H, Nederlof P M. Automated array-CGH optimized for archival formalin-fixed, paraffin-embedded tumor material. BMC Cancer. 2007 Mar. 7; 7:43.
21. Picard F, Robin S, Lavielle M, Vaisse C, Daudin J J: A statistical approach for array CGH data analysis: BMC Bioinformatics 2005, 6:27
22. Tibshirani R, Hastie T, Narasimhan B, Chu G. Diagnosis of multiple cancer types by shrunken centroids of gene expression. Proc Natl Acad Sci USA. 2002 May 14; 99(10):6567-72.
23. Eisen M B, Spellman P T, Brown P O, Botstein D. Cluster analysis and display of genome-wide expression patterns. Proc Natl Acad Sci USA. 1998 Dec. 8; 95(25):14863-8.
24. Hogervorst F B, Cornelis R S, Bout M, van Vliet M, Oosterwijk J C, Olmer R, Bakker B, Klijn J G, Vasen H F, Meijers-Heijboer H, et al. Rapid detection of BRCA1 mutations by the protein truncation test. Nat. Genet. 1995 June; 10(2):208-12.
25. Tirkkonen M, Johannsson O, Agnarsson B A, Olsson H, Ingvarsson S, Karhu R, Tanner M, Isola J, Barkardottir R B, Borg A, Kallioniemi O P. Distinct somatic genetic changes associated with tumor progression in carriers of BRCA1 and BRCA2 germ-line mutations. Cancer Res. 1997 Apr. 1; 57(7):1222-7.
26. Johannsdottir H K, Jonsson G, Johannesdottir G, Agnarsson B A, Eerola H, Arason A, Heikkila P, Egilsson V, Olsson H, Johannsson O T, Nevanlinna H, Borg A, Barkardottir R B. Chromosome 5 imbalance mapping in breast tumors from BRCA1 and BRCA2 mutation carriers and sporadic breast tumors. Int J Cancer. 2006 Sep. 1; 119(5):1052-60.
27. Lakhani S R, Van De Vijver M J, Jacquemier J, Anderson T J, Osin P P, McGuffog L, Easton D F. The pathology of familial breast cancer: predictive value of immunohistochemical markers estrogen receptor, progesterone receptor, HER-2, and TP53 in patients with mutations in BRCA1 and BRCA2. J Clin Oncol. 2002 May 1; 20(9):2310-8.
28. Loo L W, Grove D I, Williams E M, Neal C L, Cousens L A, Schubert E L, Holcomb I N, Massa H F, Glogovac J, Li C I, Malone K E, Daling J R, Delrow J J, Trask B J, Hsu L, Porter P L: Array comparative genomic hybridization analysis of genomic alterations in breast cancer subtypes. Cancer Res 2004, 64:8541-8549.
29. Fridlyand J, Snijders A M, Ylstra B, Li H, Olshen A, Segraves R, Dairkee S, Tokuyasu T, Ljung B M, Jain A N, McLennan J, Ziegler J, Chin K, Devries S, Feiler H, Gray J W, Waldman F, Pinkel D, Albertson D G. Breast tumor copy number aberration phenotypes and genomic instability. BMC Cancer. 2006 Apr. 18; 6:96.
30. Lakhani S R, Reis-Filho J S, Fulford L, Penault-Llorca F, van der Vijver M, Parry S, Bishop T, Benitez J, Rivas C, Bignon Y J, Chang-Claude J, Hamann U, Cornelisse C J, Devilee P, Beckmann M W, Nestle-Kramling C, Daly P A, Haites N, Varley J, Lalloo F, Evans G, Maugard C, Meijers-Heijboer H, Klijn J G, Olah E, Gusterson B A, Pilotti S, Radice P, Scherneck S, Sobol H, Jacquemier J, Wagner T, Peto J, Stratton M R, McGuffog L, Easton D F; Breast Cancer Linkage Consortium. Prediction of BRCA1 status in patients with breast cancer using estrogen receptor and basal phenotype. Clin Cancer Res. 2005 Jul. 15; 11(14):5175-80.
31. Turner N C, Reis-Filho J S, Russell A M, Springall R J, Ryder K, Steele D, Savage K, Gillett C E, Schmitt F C, Ashworth A, Tutt A N. BRCA1 dysfunction in sporadic basal-like breast cancer. Oncogene. 2007 Mar. 29; 26(14):2126-32.
32. Evans D G, Eccles D M, Rahman N, Young K, Bulman M, Amir E, Shenton A, Howell A, Lalloo F. A new scoring system for the chances of identifying a BRCA1/2 mutation outperforms existing models including BRCAPRO. J Med. Genet. 2004 June; 41(6):474-80.
33. Tischkowitz M, Hamel N, Carvalho M A, Birrane G, Soni A, van Beers E H, Joosse S A, Wong N, Novak D, Quenneville L A, Grist S, kConFab, Nederlof P M, Goldgar D E, Tavtigian S V, Monteir A N A, Ladias J A A, Foulkes W D. Pathogenicity of a BRCA1 missense variant is determined by the disruption of the phosphopeptide binding pocket—a milti-model approach. Submitted. 2007

TABLE 1
Definition of chromosomal regions with copy number aberrations for use in
the methods that distinguish between BRCA1-associated tumours and
sporadic tumours.

| | | | | | mid position | mid position | | |
|---|---|---|---|---|---|---|---|---|
| Chromosome | begin BAC | end BAC | | | begin | end | size Mb | aberration |
| 5 | RP11-402F5 | RP11-20O13 | 5q13.1 | 5q15 | 66792004 | 93151483 | 26.4 | loss |
| 3 | RP11-22E12 | RP11-65J14 | 3q22.1 | 3q27.2 | 135928136 | 187334918 | 51.4 | gain |
| 13 | RP11-632L2 | RP11-255P5 | 13q31.3 | 13q33.1 | 92590544 | 102357531 | 9.8 | gain |
| 12 | RP1-97G4 | RP11-478H3 | 12q21.2 | 12q23.3 | 76303262 | 104864663 | 28.6 | loss |
| 10 | RP4-542G16 | RP1-251M9 | 10p14 | 10p14 | 7315714 | 11038980 | 3.7 | gain |
| 3 | RP11-3B7 | RP11-447A21 | 3p21.31 | 3p21.1 | 49324919 | 52649659 | 3.3 | loss |
| 14 | RP11-533L7 | RP11-204K16 | 14q22.1 | 14q24.1 | 53182362 | 67909067 | 14.7 | loss |
| 6 | RP3-365E2 | RP1-153G14 | 6p23 | 6p22.1 | 14604968 | 27485724 | 12.9 | gain |
| 5 | RP11-17L14 | CTB-54G2 | 5q21.3 | 5q23.2 | 107421426 | 126761524 | 19.3 | loss |
| 1 | RP11-342M1 | RP11-14O19 | 1p34.2 | 1p21.3 | 43090968 | 95599589 | 52.5 | gain |

TABLE 2
Exemplary BAC clones that may be used to detect or generate
probes to detect copy number aberrations in the genomic
locations of the invention.

| Clone | Chromosome |
|---|---|
| RP11-342M1 | 1p34 |
| RP11-420M12 | 1p34 |
| RP11-243A18 | 1p32 |
| RP11-20F20 | 1p32 |
| RP11-5P4 | 1p31 |
| RP4-700A9 | 1p31 |
| RP5-1033K19 | 1p31 |
| RP11-250D8 | 1p31 |
| RP11-413E1 | 1p22 |
| RP11-14O19 | 1p21 |
| RP4-787H6 | 1p13 |
| RP11-98D18 | 1q21 |
| RP1-97P20 | 1q24 |
| RP5-1026E2 | 1q25 |
| RP11-469A15 | 1q32 |
| RP4-799G3 | 1q42 |
| RP11-132H1 | 2p24 |
| RP11-247H16 | 2p24 |
| RP11-298E18 | 2q14 |
| RP11-32C20 | 2q21 |
| RP11-176L20 | 2q31 |
| RP11-38H6 | 2q31 |
| RP11-378A13 | 2q35 |
| RP11-86O17 | 2q36 |
| RP11-457P23 | 2q36 |
| RP11-3B7 | 3p21 |
| RP11-89f17 | 3p21 |
| RP11-447A21 | 3p21 |
| RP11-484I19 | 3q12 |
| RP11-115B22 | 3q13 |
| RP11-324H4 | 3q13 |
| RP11-22E12 | 3q22 |
| RP11-269A14 | 3q22 |
| RP11-349D24 | 3q23 |
| RP11-349D24 | 3q23 |
| RP11-89E16 | 3q23 |
| RP11-231L11 | 3q23 |
| RP11-235I18 | 3q23 |
| RP11-160a13 | 3q24 |
| RP11-160A13 | 3q24 |
| RP11-165M11 | 3q24 |
| RP11-21M4 | 3q24 |
| RP11-251C9 | 3q25 |
| RP11-3F11 | 3q25 |
| RP11-240G5 | 3q25 |
| RP11-223L18 | 3q25 |
| RP11-6F2 | 3q25 |
| RP11-209h21 | 3q25 |
| RP11-209H21 | 3q25 |
| RP11-209H21 | 3q25 |
| RP11-203L15 | 3q26 |
| RP11-395F21 | 3q26 |
| RP11-816J6 | 3q26 |
| RP11-362K14 | 3q26 |
| RP11-163H6 | 3q26 |
| RP11-477P16 | 3q26 |
| RP11-91K9 | 3q26 |
| RP11-682A21 | 3q26 |
| RP11-420J11 | 3q26 |
| RP11-510K16 | 3q26 |
| RP11-416O18 | 3q26 |
| RP11-65J14 | 3q27 |
| RP11-324I10 | 4p16 |
| RP11-390C19 | 4p15 |
| RP11-148K14 | 4q12 |
| RP11-355L4 | 4q12 |
| RP11-19C20 | 4q21 |
| RP11-438P8 | 4q24 |
| RP11-208O6 | 4q24 |
| RP11-510D4 | 4q26 |
| RP11-148L24 | 4q34 |
| RP11-192H6 | 5p14 |
| CTD-2267H19 | 5p13 |
| RP11-34J15 | 5q12 |
| RP11-402F5 | 5q13 |
| RP11-115I6 | 5q13 |
| RP11-97L2 | 5q13 |
| RP11-241j12 | 5q14 |
| RP11-356D23 | 5q14 |
| RP11-356D23 | 5q14 |
| RP11-3H15 | 5q14 |
| RP11-12D3 | 5q14 |
| CTD-2011L22 | 5q14 |
| RP11-17L14 | 5q21 |
| RP11-249M12 | 5q23 |
| RP11-11P11 | 5q23 |
| CTB-54G2 | 5q23 |
| CTB-3C20 | 5q33 |
| RP11-511M9 | 5q34 |
| RP11-163I22 | 6p25 |
| RP3-365E2 | 6p23 |
| RP11-68J15 | 6p22 |
| RP11-408C8 | 6p22 |
| RP4-625H18 | 6p22 |
| RP3-444C7 | 6p22 |
| RP11-176J5 | 6p22 |
| RP11-289G11 | 6p22 |
| RP1-153G14 | 6p22 |
| RP11-472M19 | 6p12 |
| RP11-767J14 | 6q12 |
| RP3-429G5 | 6q21 |
| GS-57-H24 | 6q27 |
| RP11-505D17 | 7p21 |
| RP11-512E16 | 7p21 |
| RP11-126C19 | 7q31 |
| RP11-269N18 | 7q34 |
| RP4-764O12 | 7q36 |
| RP11-540E4 | 8p23 |
| RG-41-L13 | 9p24 |
| RP11-509J21 | 9p24 |
| RP11-5P15 | 9p21 |
| RP11-20P5 | 9p21 |
| RP11-336N8 | 9q21 |
| RP11-66D1 | 9q21 |
| RP11-423O13 | 9q22 |
| RP11-333I7 | 9q22 |
| RP11-23J9 | 9q31 |
| RP11-400A24 | 9q32 |
| RP11-78H18 | 9q33 |
| RP4-542G16 | 10p14 |
| RP11-566K1 | 10p14 |
| RP1-251M9 | 10p14 |
| RP11-2K17 | 10p13 |
| RP11-307B23 | 10p12 |
| RP11-505N10 | 10p11 |
| RP11-313B15 | 10q21 |
| RP11-210G22 | 10q21 |
| RP1-316D7 | 11p13 |
| RP11-291N1 | 11q14 |
| RP11-264F23 | 12p13 |
| RP11-319E16 | 12p13 |
| RP11-548L8 | 12q13 |
| RP11-366L20 | 12q14 |
| RP1-97G4 | 12q21 |
| RP11-26L7 | 12q21 |
| RP11-362A1 | 12q21 |
| RP11-87P13 | 12q21 |
| RP11-435O22 | 12q21 |
| RP11-239F20 | 12q21 |
| RP11-2K12 | 12q22 |
| RP11-435E3 | 12q22 |
| RP11-510I5 | 12q23 |
| RP11-406H4 | 12q23 |
| RP11-426H24 | 12q23 |
| RP11-210L7 | 12q23 |
| RP11-478H3 | 12q23 |
| RP11-18C24 | 12q24 |
| RP11-521L15 | 13q21 |
| RP11-632L2 | 13q31 |
| RP11-74A12 | 13q32 |
| RP11-235O20 | 13q32 |
| RP11-383H17 | 13q32 |
| RP11-442I9 | 13q32 |
| RP11-279D17 | 13q32 |
| RP11-118F16 | 13q33 |
| RP11-564N10 | 13q33 |
| RP11-255P5 | 13q33 |
| RP11-310D8 | 13q34 |
| RP11-468E2 | 14q12 |
| RP11-34O18 | 14q21 |
| RP11-332O9 | 14q21 |
| RP11-484F16 | 14q22 |
| RP11-66E7 | 14q23 |
| RP11-204K16 | 14q24 |
| RP11-368K8 | 14q24 |
| RP11-406A9 | 14q31 |
| RP11-179O11 | 14q31 |
| RP11-365N19 | 14q32 |
| RP11-13O24 | 15p13 |
| RP11-380D11 | 15q15 |
| RP11-151N17 | 15q21 |
| RP11-154J22 | 15q21 |
| RP11-266O8 | 15q26 |
| RP11-152P23 | 16p13 |
| RP11-31O11 | 16p13 |
| RP11-368N21 | 16p11 |
| RP11-424K7 | 16q12 |
| RP11-481J2 | 16q13 |
| RP11-105C20 | 16q21 |
| RP11-411B10 | 18p11 |
| RP11-45A1 | 18q22 |
| RP11-268O21 | 19p13 |
| RP4-796I11 | 20q12 |
| RP11-304D2 | 21q21 |
| RP1-245P17 | 21q22 |
| RP1-255P7 | 21q22 |
| RP11-98O13 | 21q22 |
| CTA-397C4 | 22q13 |
| RP11-445O16 | 23p11 |
| RP3-394F12 | 23q25 |
| RP3-428A13 | 23q25 |

TABLE 3
Pathological characteristics of the analysed BRCA1 mutation
carriers, sporadic, and HBOC breast carcinomas.

| | BRCA1 | Sporadic | HBOC |
|---|---|---|---|
| No. analysed | 28 | 48 | 48 |
| ER+ | 3.6% (1/28) | 54.3% (25/46) | 68.9% (31/45) |
| PR+ | 3.6% (1/28) | 46.8% (22/47) | 50.0% (23/46) |
| Her2/Neu+ | 3.8% (1/26) | 40.0% (17/46) | 9.8% (4/41) |
| TP53+ | 44.4% (12/27) | 43.5% (20/46) | 9.8% (4/41) |

ER, estrogen receptor; PR, progesterone receptor; +, positive.

TABLE 4
LOH PCR primers.

| Marker | Forward Primer | Reverse Primer |
|---|---|---|
| D17S1322 | CTAGCCTGGGCAACAAACGA | GCAGGAAGCAGGAATGGAAC |
| D17S1323 | TAGGAGATGGATTATTGGTG | AAGCAACTTTGCAATGAGTG |
| D17S588 | CCTGGTCTAGGAAGAGTGTCA | GTGTAAGCATCTGTGTATACTAC |
| D17S578 | CTATCAATAAGCATTGGCCT | CTGGAGTTGAGACTAGCCT |
| THRA1 | CTGCGCTTTGCACTATTGGG | GTGTCTTCGGGCAGCATAGCATTGCCT |

TABLE 5
LOH PCR Program

| | | | |
|---|---|---|---|
| 1 | 5 min 94° | C. | |
| 2 | 15 sec 94° | C. | 10x |
| 3 | 15 sec 55° | C. | |
| 4 | 30 sec 72° | C. | |
| 5 | 15 sec 89° | C. | 20x |
| 6 | 15 sec 55° | C. | |
| 7 | 30 sec 72° | C. | |
| 8 | 10 min 72° | C. | |
| 9 | ∞ 15° | C. | |

TABLE 6
Mean frequency and locations of the aberrations of 28 BRCA1-
associated and 48 sporadic breast tumours for high frequency
(>20%) aberrant chromosomal regions (>10 Mb).

| | | BRCA1 | Sporadic | |
|---|---|---|---|---|
| Chr | aberration | percentage | percentage | p-value |
| 1q | gain | 43.0 | 36.9 | <0.001 |
| 3q22-29 | gain | 33.6 | 5.8 | <0.001 |
| 5q11-15 | loss | 24.2 | 3.6 | <0.001 |
| 6p25-22 | gain | 30.9 | 3.5 | <0.001 |
| 7q31-36 | gain | 26.1 | 4.0 | <0.001 |
| 8p23 | loss | 23.4 | 19.7 | 0.040 |
| 8q | gain | 47.1 | 31.7 | <0.001 |
| 9p21-11 | loss | 30.6 | 21.2 | <0.001 |
| 10p15-14 | gain | 52.5 | 27.7 | <0.001 |
| 10p14-13 | gain | 29.6 | 11.0 | <0.001 |
| 11p14-13 | gain | 20.7 | 4.9 | <0.001 |
| 11q22-25 | loss | 8.9 | 22.4 | <0.001 |
| 12p13-12 | gain | 29.2 | 8.2 | <0.001 |
| 13q | loss | 33.0 | 18.5 | <0.001 |
| 13q32-34 | gain | 20.5 | 1.4 | <0.001 |
| 14q22-23 | loss | 32.1 | 7.7 | <0.001 |
| 14q32 | loss | 36.3 | 9.5 | <0.001 |
| 15q11-12 | loss | 35.7 | 28.3 | 0.007 |
| 15q14-21 | loss | 23.7 | 3.5 | <0.001 |
| 16q | loss | 5.0 | 27.2 | <0.001 |
| 17p | loss | 9.7 | 22.8 | <0.001 |
| 17q22-23 | gain | 23.8 | 20.5 | 0.082 |
| 17q25 | gain | 21.4 | 11.2 | <0.001 |
| 18q21-22 | gain | 24.3 | 6.0 | <0.001 |
| 22q13 | loss | 25.0 | 21.5 | 0.303 |
| 23p22 | loss | 21.0 | 9.4 | <0.001 |
| 23p11 | loss | 27.3 | 9.7 | <0.001 |
| 23q | loss | 32.7 | 14.9 | <0.001 |

P-values for the significance in aberration difference between tumour groups.

TABLE 7
A. Patient information from the BRCA1-acossiated patients.

| | Age of | | | | | | | Classification |
|---|---|---|---|---|---|---|---|---|
| BRCA1 | diagnosis | Grade | ER | PR | Her2/Neu | TP53 | BRCA1 mutation | score |
| B127 | 39 | | − | − | − | + | c.5382insC | 21.1 (train) |
| B126 | 40 | | − | − | − | + | c.IVS21 − 36del510 | 4.0 (validation) |
| B135 | 41 | | − | − | − | + | c.2312del5 | 7.1 (train) |
| B137 | 32 | | − | − | − | − | c.IVS12 − 1632del3835 | 9.1 (train) |
| B124 | 61 | III | − | − | − | + | c.3875del4 | 5.3 (validation) |
| B125 | 35 | | − | − | − | − | c.2804delAA | 4.3 (train) |
| B141 | 39 | | + | + | − | − | c.IVS21 − 36del510 | 1.6 (validation) |
| B107 | 41 | II | − | − | − | − | c.1319delT | 3.0 (train) |
| B108 | 44 | II | − | − | | | c.1411insT | 23.4 (train) |
| B109 | 30 | III | − | − | − | − | c.IVS21 − 36del510 | 6.0 (train) |
| B145 | 33 | III | − | − | − | − | c.185delAG | 5.3 (train) |
| B146 | 33 | III | − | − | − | − | c.185delAG | 9.4 (validation) |
| B149 | 31 | III | − | − | − | + | c.IVS20 + 1G > A | 18.0 (train) |
| B150 | 41 | III | − | − | − | − | c.IVS21 − 36del510 | 6.4 (validation) |
| B152 | 47 | III | − | − | − | + | c.IVS13 + 4123ins6081 | 7.0 (validation) |
| B153 | 48 | III | − | − | | + | c.185delAG | 13.4 (train) |
| B116 | 49 | | − | − | − | + | c.185delAG | 17.7 (train) |
| B156 | 47 | II-III | − | − | − | + | c.5382insC | 5.3 (validation) |
| B118 | 34 | III | − | − | − | − | c.4416_4417delTTinsG | 23.4 (train) |
| B119 | 34 | III | − | − | − | − | c.4416_4417delTTinsG | 14.7 (valid) |
| B158 | 27 | III | − | − | − | + | c.IVS21 − 36del510 | 10.8 (train) |
| B160 | 61 | III | − | − | − | − | c.IVS20 + 1G > A | 8.0 (train) |
| B161 | 30 | III | − | − | − | + | c.IVS13 + 4123ins6081 | 4.1 (validation) |
| B122 | 45 | III | − | − | − | − | c.4446C > T | 8.0 (train) |
| B162 | 27 | | − | − | − | − | c.IVS20 + 1G > A | 1.7 (validation) |
| B164 | 31 | III | − | − | − | − | c.del exonen 1A-7 | 8.8 (train) |
| B165 | 33 | III | − | − | − | + | c.IVS12 − 1632del3835 | 4.4 (train) |
| B171 | 33 | III | − | − | + | − | c.IVS2 − 9C > G | 7.4 (train) |

B. Patient information from the sporadic patients.

| | Age of | | | | | | Classification |
|---|---|---|---|---|---|---|---|
| Sporadic | diagnosis | Grade | ER | PR | Her2/Neu | TP53 | score |
| C001 | 38 | II | − | + | − | − | −7.8 (train) |
| C002 | 45 | II | − | − | + | − | −9.3 (validation) |
| C004 | 49 | III | − | − | + | + | −0.4 (train) |
| C006 | 37 | III | − | − | + | − | −10.9 (valid) |
| C010 | 40 | | + | + | − | − | −9.6 (train) |
| C013 | 48 | III | − | + | + | + | −11.5 (valid) |
| C015 | 49 | III | + | − | + | + | −10.5 (train) |
| C016 | 48 | | + | + | − | − | −13.6 (train) |
| C017 | 36 | III | + | + | − | − | −14.7 (train) |
| C018 | 44 | III | − | − | − | − | −1.5 (train) |
| C019 | 47 | II | + | + | − | − | −11.1 (train) |
| C020 | 34 | III | − | − | − | + | −3.8 (valid) |
| C022 | 51 | | + | − | − | − | −11.0 (valid) |
| C023 | 45 | II | + | + | + | − | −1.7 (validation) |
| C025 | 50 | I-II | + | + | − | − | −7.8 (train) |
| C026 | 37 | I-II | + | + | − | − | −10.1 (train) |
| C027 | 45 | I-II | + | + | + | − | −11.4 (train) |
| C028 | 41 | | + | + | − | − | −11.5 (valid) |
| C029 | 50 | | − | − | + | − | −8.5 (validation) |
| C030 | 38 | | + | − | − | − | −9.9 (train) |
| C031 | 43 | | − | − | + | + | −13.6 (train) |
| C032 | 46 | | + | + | − | − | −11.2 (train) |
| C033 | 45 | | − | − | − | + | −3.4 (train) |
| C034 | 47 | III | + | + | − | − | −12.6 (train) |
| C035 | 53 | | | | | | −7.9 (train) |
| C036 | 40 | I-II | + | + | + | − | −11.0 (train) |
| C037 | 49 | | + | + | − | − | −17.0 (train) |
| C039 | 41 | III | | + | | | −7.4 (validation) |
| C042 | 42 | II-III | + | + | − | − | −14.5 (valid) |
| C043 | 32 | | − | − | + | − | −7.1 (validation) |
| C044 | 45 | | + | − | − | − | −10.5 (train) |
| C046 | 34 | III | − | − | − | + | −6.8 (train) |
| C047 | 39 | III | − | − | − | + | −0.1 (train) |
| C048 | 33 | | − | − | + | + | −9.1 (validation) |
| C049 | 39 | III | − | − | − | + | −1.9 (validation) |
| C051 | 44 | I-II | − | − | + | − | −15.4 (train) |
| C052 | 45 | | − | + | − | − | −13.5 (train) |
| C053 | 42 | II-III | − | − | − | + | −2.5 (train) |
| C056 | 48 | III | − | − | − | + | −2.3 (train) |
| C057 | 51 | | − | − | − | − | −13.1 (valid) |
| C058 | 49 | II | + | − | + | + | −6.8 (train) |
| C060 | 47 | | + | + | − | + | −14.5 (train) |
| C061 | 41 | II | + | + | + | + | −6.5 (train) |
| C063 | 46 | III | + | + | − | + | −1.0 (validation) |
| C065 | 48 | III | − | − | − | + | −2.9 (train) |
| C067 | 62 | II | + | + | + | + | −2.5 (validation) |
| C068 | 56 | III | + | − | + | + | −2.4 (train) |
| C069 | 60 | III | + | − | − | + | −2.5 (train) |

C. Patient information from the HBOC patients.

| | | | | | | | Evans | |
|---|---|---|---|---|---|---|---|---|
| | Age of | | | | | | Score | Classification |
| HBOC | diagnosis | Grade | ER | PR | Her2/Neu | TP53 | BRCA1 | score |
| HBC41 | 57 | II | + | + | − | − | 11.8% | 4.9 |
| HBC34 | 51 | I-II | − | − | − | − | 20.0% | 4.1 |
| HBC17 | 54 | II | + | − | − | − | | 1.6 |
| HBC15 | 20 | | − | − | − | − | 36.0% | 0.2 |
| HBC28 | 46 | I-II | + | + | − | − | | 0.0 |
| HBC16 | 46 | I | + | + | − | − | 36.0% | 0.1 |
| HBC22 | 37 | | − | − | − | − | 50.0% | −1.1 |
| HBC14 | 42 | | − | − | − | − | 11.8% | −1.3 |
| HBC18 | 55 | II | − | + | − | − | 36.0% | −1.8 |
| HBC31 | 51 | III | − | − | | | 36.0% | −2.0 |
| HBC40 | 53 | II | + | − | − | + | 20.0% | −2.1 |
| HBC26 | 52 | II | + | + | − | − | | −2.2 |
| HBC07 | 54 | III | + | − | + | − | 20.0% | −2.7 |
| HBC23 | 47 | | + | − | − | − | 11.8% | −2.7 |
| HBC13 | 51 | II | + | + | − | − | 11.8% | −3.0 |
| HBC35 | 36 | | − | − | − | − | 78.0% | −3.1 |
| HBC03 | 55 | II | − | − | − | + | 11.8% | −3.3 |
| HBC09 | 54 | II-III | | − | + | − | | −3.5 |
| HBC19 | 53 | | − | − | − | + | 1.4% | −3.6 |
| HBC25 | 52 | | + | + | − | − | | −4.3 |
| HBC06 | 59 | II | + | − | − | − | 11.8% | −5.3 |
| HBC02 | 25 | II | − | − | | | 36.0% | −5.5 |
| HBC11 | 44 | II | + | + | | | 36.0% | −5.9 |
| HBC36 | 60 | I-II | + | + | − | − | 20.0% | −5.9 |
| HBC10 | 44 | II | + | + | | | 36.0% | −6.3 |
| HBC44 | 52 | II | + | + | + | − | 20.0% | −6.6 |
| HBC39 | 55 | I | + | − | | − | 20.0% | −6.6 |
| HBC47 | 46 | I | | | | | 20.0% | −6.7 |
| HBC29 | 47 | II-III | + | + | − | − | 1.4% | −6.8 |
| HBC45 | 43 | II | − | − | − | − | 36.0% | −8.5 |
| HBC12 | 37 | | + | + | − | − | 3.8% | −8.5 |
| HBC24 | 39 | | − | − | − | − | 50.0% | −8.9 |
| HBC33 | 46 | II-III | + | + | − | − | 78.0% | −9.0 |
| HBC48 | 43 | III | − | − | − | | 36.0% | −9.1 |
| HBC27 | 58 | II-III | | | | | 50.0% | −9.7 |
| HBC46 | 45 | II | + | + | − | − | 36.0% | −9.8 |
| HR0bc8 | 47 | II | + | + | − | − | 11.8% | −9.9 |
| HBC38 | 61 | II | + | − | − | − | | −10.1 |
| HBC30 | 37 | I-II | + | + | − | − | 20.0% | −10.1 |
| HBC42 | 37 | III | + | + | − | − | 1.4% | −10.4 |
| HBC21 | 56 | | + | − | − | − | 20.0% | −10.5 |
| HBC04 | 61 | | − | − | − | − | | −10.6 |
| HBC32 | 44 | II-III | + | + | + | − | 78.0% | −11.2 |
| HBC37 | 47 | I | + | + | − | − | 3.8% | −11.6 |
| HBC43 | 52 | I-II | + | + | − | − | | −12.0 |
| HBC05 | 58 | I | + | + | − | − | 11.8% | −12.1 |
| HBC20 | 44 | | + | + | − | − | 36.0% | −12.6 |
| HBC01 | 57 | | + | − | − | + | 3.8% | −15.1 |

1. A method for determining whether cells in a sample are from a BRCA1-related tumour or from a sporadic tumour, comprising detecting the number of copies per cell in genomic DNA of at least three of the following genomic locations: 1p21-34, 3p21, 3q22-27, 5q13-15, 5q21-23, 6p22-23, 10p14, 12q21-23, 13q31-33 or 14q22-24,
wherein detection of:
(i) an increase in the number of copies per cell of DNA in genomic locations 1p21-34, 3q22-27, 6p22-23, 10p14, or 13g31-33; and/or
(ii) a decrease in the number of copies per cell of DNA in genomic locations 3p21, 5q13-15, 5q21-23, 12q21-23, or 14g22-24,
compared to the number of copies per cell of DNA in said genomic locations in non-cancer cells, indicates that the cell sample is from a BRCA1-related tumour.

2. The method according to claim 1, wherein the detection is quantitative or semi-quantitative.

3. The method according to claim 1, wherein the number of copies per cell of genomic DNA at least six of said genomic locations is detected.

4. The method according to claim 1, wherein the genomic locations comprise at least 5q13-15, 3q22-27 and 13q31-33.

5. The method according claim 1, further comprising detecting the number of copies per cell of genomic DNA in the cell sample at least one of 5q13-15, 3q22-27 and 13q31-33,
wherein a decrease in the number of copies per cell of DNA in 5q13-15, 3q22-27 and 13q31-33 compared to the number of copies per cell of DNA in said genomic locations in non-cancer cells, indicates that the cell sample is from a sporadic tumor.

6. The method according to claim 1, wherein the number of copies per cell of the genomic locations is detected by hybridization to a probe comprising at least 10 contiguous nucleotides of a sequence that is present in one of the following BAC clones: RP11-402F5-RP11-20O13, RP11-22E12-RP11-65J14, RP11-632L2-RP11-255P5, RP1-97G4-RP11-478H3, RP4-542G16-RP1-251M9, RP11-3B7-RP11-447A21, RP11-533L7-RP11-204K16, RP3-365E2-RP1-153G14, RP11-17L14-CTB-54G2, RP11-342M1-RP11-14O19, RP11-342M1, RP11-148K14, RP11-291N1, RP11-420M12, RP11-355L4, RP11-264F23, RP11-243A18, RP11-19C20, RP11-319E16, RP11-20F20, RP11-438P8, RP11-548L8, RP11-5P4, RP11-208O6, RP11-366L20, RP4-700A9, RP11-510D4, RP1-97G4, RP5-1033K19, RP11-148L24, RP11-26L7, RP11-250D8, RP11-192H6, RP11-362A1, RP11-413E1, CTD-2267H19, RP11-87P13, RP11-14O19, RP11-34J15, RP11-435O22, RP4-787H6, RP11-402F5, RP11-239F20. RP11-98D18, RP11-115I6, RP11-2K12, RP1-97P20, RP11-97L2, RP11-435E3, RP5-1026E2, RP11-241j12, RP11-510I5, RP11-469A15, RP11-356D23, RP11-406H4, RP4-799G3, RP11-356D23, RP11-426H24, RP11-132H1, RP11-3H15, RP11-210L7, RP11-247H16, RP11-12D3, RP11-478H3, RP11-298E18, CTD-2011L22, RP11-18C24, RP11-32C20, RP11-17L14, RP11-521L15, RP11-176L20, RP11-249M12, RP11-632L2, RP11-38H6, RP11-11P11, RP11-74A12, RP11-378A13, CTB-54G2, RP11-235O20, RP11-86O17, CTB-3C20, RP11-383H17, RP11-457P23, RP11-511M9, RP11-442I9, RP11-3B7, RP11-163I22, RP11-279D17, RP11-89f17, RP3-365E2, RP11-118F16, RP11-447A21, RP11-68J15, RP11-564N10, RP11-484I19, RP11-408C8, RP11-255P5, RP11-115B22, RP4-625H18, RP11-310D8, RP11-324H4, RP3-444C7, RP11-468E2, RP11-22E12, RP11-176J5, RP11-34O18, RP11-269A14, RP11-289G11, RP11-332O9, RP11-349D24, RP1-153G14, RP11-484F16, RP11-349D24, RP11-472M19, RP11-66E7, RP11-89E16, RP11-767J14, RP11-204K16, RP11-231L11, RP3-429G5, RP11-368K8, RP11-235I18, GS-57-H24, RP11-406A9, RP11-160a13, RP11-505D17, RP11-179O11, RP11-160A13, RP11-512E16, RP11-365N19, RP11-165M11, RP11-126C19, RP11-13O24, RP11-21M4, RP11-269N18, RP11-380D11, RP11-251C9, RP4-764O12, RP11-151N17, RP11-3F11, RP11-540E4, RP11-154J22, RP11-240G5, RG-41-L13, RP11-266O8, RP11-223L18, RP11-509J21, RP11-152P23, RP11-6F2, RP11-5P15, RP11-31O11, RP11-209h21, RP11-20P5, RP11-368N21, RP11-209H21, RP11-336N8, RP11-424K7, RP11-209H21, RP11-66D1, RP11-481J2, RP11-203L15, RP11-423O13, RP11-105C20, RP11-395F21, RP11-333I7, RP11-411B10, RP11-816J6, RP11-23J9, RP11-45A1, RP11-362K14, RP11-400A24, RP11-268O21, RP11-163H6, RP11-78H18, RP4-796I11, RP11-477P16, RP4-542G16, RP11-304D2, RP11-91K9, RP11-566K1, RP1-245P17, RP11-682A21, RP1-251M9, RP1-255P7, RP11-420J11, RP11-2K17, RP11-98O13, RP11-510K16, RP11-307B23, CTA-397C4, RP11-416O18, RP11-505N10, RP11-445O16, RP11-65J14, RP11-313B15, RP3-394F12, RP11-324I10, RP11-210G22, RP3-428A13, RP11-390C19, and RP1-316D7.

7. A set of at least three nucleic acid probes, each of which specifically hybridizes to a different genomic location selected from the group consisting of 1p21-34, 3p21, 3q22-27, 5q13-15, 5q21-23, 6p22-23, 10p14, 12q21-23, 13q31-33 and 14q22-24.

8. The set of nucleic acid probes according to claim 7, that comprises at least six of said probes, wherein each probe specifically hybridizes to a different one of said genomic locations.

9. The set of nucleic acid probes according to claim 7, wherein each probe comprises at least 10 contiguous nucleotides of a sequence that is present in one of the following BAC clones: RP11-402F5-RP11-20O13, RP11-22E12-RP11-65J14, RP11-632L2-RP11-255P5, RP1-97G4-RP11-478H3, RP4-542G16-RP1-251M9, RP11-3B7-RP11-447A21, RP11-533L7-RP11-204K16, RP3-365E2-RP1-153G14, RP11-17L14-CTB-54G2, RP11-342M1-RP11-14O19, RP11-342M1, RP11-148K14, RP11-291N1, RP11-420M12, RP11-355L4, RP11-264F23, RP11-243A18, RP11-19C20, RP11-319E16, RP11-20F20, RP11-438P8, RP11-548L8, RP11-5P4, RP11-208O6, RP11-366L20, RP4-700A9, RP11-510D4, RP1-97G4, RP5-1033K19, RP11-148L24, RP11-26L7, RP11-250D8, RP11-192H6, RP11-362A1, RP11-413E1, CTD-2267H19, RP11-87P13, RP11-14O19, RP1′-34J15, RP11-435O22, RP4-787H6, RP11-402F5, RP11-239F20. RP11-98D18, RP11-115I6, RP11-2K12, RP1-97P20, RP11-97L2, RP11-435E3, RP5-1026E2, RP11-241j12, RP11-510I5, RP11-469A15, RP11-356D23, RP11-406H4, RP4-799G3, RP11-356D23, RP11-426H24, RP11-132H1, RP11-3H15, RP11-210L7, RP11-247H16, RP11-12D3, RP11-478H3, RP11-298E18, CTD-2011L22, RP11-18C24, RP11-32C20, RP11-17L14, RP11-521L15, RP11-176L20, RP11-249M12, RP11-632L2, RP11-38H6, RP11-11P11, RP11-74A12, RP11-378A13, CTB-54G2, RP11-235O20, RP11-86O17, CTB-3C20, RP11-383H17, RP11-457P23, RP11-511M9, RP11-442I9, RP11-3B7, RP11-163I22, RP11-279D17, RP11-89f17, RP3-365E2, RP11-118F16, RP11-447A21, RP11-68J15, RP11-564N10, RP11-484I19, RP11-408C8, RP11-255P5, RP11-115B22, RP4-625H18, RP11-310D8, RP11-324H4, RP3-444C7, RP11-468E2, RP11-22E12, RP11-176J5, RP11-34O18, RP11-269A14, RP11-289G11, RP11-332O9, RP11-349D24, RP1-153G14, RP11-484F16, RP11-349D24, RP11-472M19, RP11-66E7, RP11-89E16, RP11-767J14, RP11-204K16, RP11-231L11, RP3-429G5, RP11-368K8, RP11-235I18, GS-57-H24, RP11-406A9, RP11-160a13, RP11-505D17, RP11-179O11, RP11-160A13, RP11-512E16, RP11-365N19, RP11-165M11, RP11-126C19, RP11-13O24, RP11-21M4, RP11-269N18, RP11-380D11, RP11-251C9, RP4-764O12, RP11-151N17, RP11-3F11, RP11-540E4, RP11-154J22, RP11-240G5, RG-41-L13, RP11-266O8, RP11-223L18, RP11-509J21, RP11-152P23, RP11-6F2, RP11-5P15, RP11-31O11, RP11-209h21, RP11-20P5, RP11-368N21, RP11-209H21, RP11-336N8, RP11-424K7, RP11-209H21, RP11-66D1, RP11-481J2, RP11-203L15, RP11-423O13, RP11-105C20, RP11-395F21, RP11-333I7, RP11-411B10, RP11-816J6, RP11-23J9, RP11-45A1, RP11-362K14, RP11-400A24, RP11-268O21, RP11-163H6, RP11-78H18, RP4-796I11, RP11-477P16, RP4-542G16, RP11-304D2, RP11-91K9, RP11-566K1, RP1-245P17, RP11-682A21, RP1-251M9, RP1-255P7, RP11-420J11, RP11-2K17, RP11-98O13, RP11-510K16, RP11-307B23, CTA-397C4, RP11-416O18, RP11-505N10, RP11-445O16, RP11-65J14, RP11-313B15, RP3-394F12, RP11-324I10, RP11-210G22, RP3-428A13, RP11-390C19, and RP1-316D7.

10. The set of nucleic acid probes according to claim 9, wherein each probe comprises a sequence that is unique in the genome.

11. The set of nucleic acid probes according to claim 9, wherein the sequences of at least two of the probes are selected from the group of sequences SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, and SEQ ID NO:10.

12. A set of at least three BAC clones selected from the group consisting of RP11-402F5-RP11-20O13, RP11-22E12-RP11-65J14, RP11-632L2-RP11-255P5, RP1-97G4-RP11-478H3, RP4-542G16-RP1-251M9, RP11-3B7-RP11-447A21, RP11-533L7-RP11-204K16, RP3-365E2-RP1-153G14, RP11-17L14-CTB-54G2, RP11-342M1-RP11-14O19, RP11-342M1, RP11-148K14, RP11-291N¹, RP11-420M12, RP11-355L4, RP11-264F23, RP11-243A18, RP11-19C20, RP11-319E16, RP11-20F20, RP11-438P8, RP11-548L8, RP11-5P4, RP11-208O6, RP11-366L20, RP4-700A9, RP11-510D4, RP1-97G4, RP5-1033K19, RP11-148L24, RP11-26L7, RP11-250D8, RP11-192H6, RP11-362A1, RP11-413E1, CTD-2267H19, RP11-87P13, RP11-14O19, RP11-34J15, RP11-435O22, RP4-787H6, RP11-402F5, RP11-239F20. RP11-98D18, RP11-115I6, RP11-2K12, RP1-97P20, RP11-97L2, RP11-435E3, RP5-1026E2, RP11-241j12, RP11-510I5, RP11-469A15, RP11-356D23, RP11-406H4, RP4-799G3, RP11-356D23, RP11-426H24, RP11-132H1, RP11-3H15, RP11-210L7, RP11-247H16, RP11-12D3, RP11-478H3, RP11-298E18, CTD-2011L22, RP11-18C24, RP11-32C20, RP11-17L14, RP11-521L15, RP11-176L20, RP11-249M12, RP11-632L2, RP11-38H6, RP11-11P11, RP11-74A12, RP11-378A13, CTB-54G2, RP11-235O20, RP11-86O17, CTB-3C20, RP11-383H17, RP11-457P23, RP11-511M9, RP11-442I9, RP11-3B7, RP11-163I22, RP11-279D17, RP11-89f17, RP3-365E2, RP11-118F16, RP11-447A21, RP11-68J15, RP11-564N10, RP11-484I19, RP11-408C8, RP11-255P5, RP11-115B22, RP4-625H18, RP11-310D8, RP11-324H4, RP3-444C7, RP11-468E2, RP11-22E12, RP11-176J5, RP11-34O18, RP11-269A14, RP11-289G11, RP11-332O9, RP11-349D24, RP1-153G14, RP11-484F16, RP11-349D24, RP11-472M19, RP11-66E7, RP11-89E16, RP11-767J14, RP11-204K16, RP11-231L11, RP3-429G5, RP11-368K8, RP11-235I18, GS-57-H24, RP11-406A9, RP11-160a13, RP11-505D17, RP11-179O11, RP11-160A13, RP11-512E16, RP11-365N19, RP11-165M11, RP11-126C19, RP11-13O24, RP11-21M4, RP11-269N18, RP11-380D11, RP11-251C9, RP4-764O12, RP11-151N17, RP11-3F11, RP11-540E4, RP11-154J22, RP11-240G5, RG-41-L13, RP11-266O8, RP11-223L18, RP11-509J21, RP11-152P23, RP11-6F2, RP11-5P15, RP11-31O11, RP11-209h21, RP11-20P5, RP11-368N21, RP11-209H21, RP11-336N8, RP11-424K7, RP11-209H21, RP11-66D1, RP11-481J2, RP11-203L15, RP11-423O13, RP11-105C20, RP11-395F21, RP11-333I7, RP11-411B10, RP11-816J6, RP11-23J9, RP11-45A1, RP11-362K14, RP11-400A24, RP11-268O21, RP11-163H6, RP11-78H18, RP4-796I11, RP11-477P16, RP4-542G16, RP11-304D2, RP11-91K9, RP11-566K1, RP1-245P17, RP11-682A21, RP1-251M9, RP1-255P7, RP11-420J11, RP11-2K17, RP11-98O13, RP11-510K16, RP11-307B23, CTA-397C4, RP11-416O18, RP11-505N10, RP11-445O16, RP11-65J14, RP11-313B15, RP3-394F12, RP11-324I10, RP11-210G22, RP3-428A13, RP11-390C19, and RP1-316D7.

13. An array comprising a positionally-addressable set of nucleic acid probes according to claim 7.

14. The array according to claim 13, wherein the probes are immobilized to a solid support.

15. A kit comprising:
(a) a set of nucleic acid probes according to claim 7, and
(b) an assay reagent or buffer for use in a method for determining whether cells in are from a BRCA1-related tumor or from a sporadic tumor.

16. A kit comprising:
(a) a set of nucleic acid probes according to claim 7, and
(b) instructions for performing a method-of determining whether cells are from a BRCA1-related tumor or from a sporadic tumor.

17. (canceled)

18. The method according to claim 3, wherein the number of copies per cell of genomic DNA at all 10 of said genomic locations is detected.

19. A kit comprising
(a) an array according to claim 13, and
(b) an assay reagent or buffer for use in a method for determining whether cells in are from a BRCA1-related tumor or from a sporadic tumor.

20. A kit comprising:
(a) an array according to claim 13, and
(b) instructions for performing a method of determining whether cells are from a BRCA1-related tumor or from a sporadic tumor.
